(19) United States
(12) Patent Application Publication
(10) Pub. No.: US 2017/0369495 A1
(43) Pub. Date: Dec. 28, 2017

(54) MOLECULAR ISOTOPIC ENGINEERING

(71) Applicant: John P. JASPER, Niantic, CT (US)

(72) Inventor: John P. JASPER, Niantic, CT (US)

(21) Appl. No.: 15/540,075

(22) PCT Filed: Dec. 30, 2015
(86) PCT No.: PCT/US2015/068007

§ 371 Date: Jun. 27, 2017

Related U.S. Application Data
(60) Provisional application No. 62/097,918, filed on Dec. 30, 2014.
(60) Provisional application No. 62/135,070, filed on Mar. 18, 2015.
(60) Provisional application No. 62/171,606, filed on Jun. 5, 2015.
(60) Provisional application No. 62/201,183, filed on Aug. 5, 2015.

(51) Int. Cl. C07D 487/04; C07C 57/32; C07C 51/41

(52) U.S. Cl. CPC C07D 487/04; C07C 51/41; C07C 57/32

(57) ABSTRACT

The present invention relates to molecular isotopic engineering. The present invention relates to a method or process for preparing a target compound of a statistically defined isotopic composition comprising the step of reacting one or more reactant compounds, wherein each reactant compound is of a statistically defined isotopic composition. The reactant compound is reacted in a chemical process or a biological process thereby generating an isotopic mass balance, or further, an isotopic fractionation to produce the target compound. The present invention also relates to a statistically defined isotopic composition of a target compound. The statistically defined isotopic composition comprises an internal marker, and can be used as, for example, a security feature, an identity indicator, or a purity indicator of the target compound.

### RELATED APPLICATIONS

This application claims priority to U.S. Provisional Patent Application Ser. No. 62/097,915 filed on Dec. 30, 2014, U.S. Provisional Patent Application Ser. No. 62/135,070 filed on Mar. 18, 2015, U.S. Provisional Patent Application Ser. No. 62/171,606 filed on Jun. 5, 2015, and U.S. Provisional Patent Application Ser. No. 62/201,183 filed on Aug. 5, 2015, the disclosures of each of which are incorporated by reference herein in their entirety.

### FIELD OF THE INVENTION

The present invention relates to molecular isotopic engineering. The present invention relates to a method or process for preparing a target compound of a statistically defined isotopic composition comprising the step of reacting one or more reactant compounds, wherein each reactant compound is of a statistically defined isotopic composition. The reactant compound is reacted in a chemical process or a biological process thereby generating an isotopic mass balance, or further, an isotopic fractionation to produce the target compound. The present invention also relates to a statistically defined isotopic composition of a target compound. The statistically defined isotopic composition comprises an internal marker, and can be used as, for example, a security feature, an identity indicator, or a purity indicator of the target compound.

### BACKGROUND OF THE INVENTION

A wide range of chemical and biological processes are used to manufacture chemicals, pharmaceuticals, biologics, polymers, food stuffs, fuels, and most other products in the modern world. However, it would be useful to have methods to not only manufacture these materials at the molecular level, i.e. to synthesize their chemical framework, but to also have methods to reproducibly define or select the isotopic composition of these materials. In other words, it would be useful to develop methods of “molecular isotopic engineering” for making products having statistically defined isotopic compositions. Such “molecularly isotopically engineered” products would essentially have an internal marker that would not interfere with or disturb the chemical composition of the underlying molecule. These engineered products would, from a molecular standpoint, be in stark contrast to bulk materials that are extrinsically doped or tagged with isotopes, or to compounds that have been positionally labelled or substituted with an isotope. An example of such bulk labelling would be the addition of a small amount of a ¹³C labelled amino acid added as an excipient to a drug product formulation. An example of positional isotopic labelling would be the substitution of a hydrogen in a molecule with a stable isotope such as a deuterium, or even with a radioactive isotope such as a tritium; to give the deuterium or tritium labelled compound. In contrast to extrinsically isotopically modified bulk products or positionally labelled compounds, those prepared by the methods of the present invention would comprise an internal marker—essentially an isotopic “bar code”—that can serve a wide variety of functions including, for example, as a security or anti-counterfeiting feature, an identity indicator, a source indicator, a process indicator, or a purity indicator, or that could serve as a proxy for a variety of chemical and physical features of the compound.

The present invention describes molecular isotopic engineering as a manufacturing method by which the stable-isotopic composition of chemical products is designed by selection of reagents (i.e., starting materials and synthetic intermediates) of known and desired isotopic composition and of selection of certain synthetic pathways. Thus, selected starting materials subject to specified conditions will generate products with a statistically-delimited isotopic range. Purposeful variations of the isotopic compositions of the starting material and/or of the isotopically-fractionating reaction conditions (e.g., reaction rate via temperature, pressure, time, reagent concentration, etc.) can be used to predetermine the isotopic ranges for the desired product.

Large-soak scientific research involving both radioactive and non-radioactive (i.e. stable) isotopes goes back to the time of the Manhattan Project, which produced the first atomic bombs during World War II. Isotopes—whether stable or radioactive—are forms of the same chemical element having different atomic masses. For example, uranium has an isotopic form with a mass of 235 (uranium-235 or ²³⁵U), and also an isotopic form with a mass of 238 (uranium-238 or ²³⁸U). Although radioactive isotopes can be used in the compositions, methods, and systems of the present invention, the compositions, methods, and systems herein are focused primarily on non-radioactive, stable isotopes.

By 1942, isotopes had only been known for about thirty years. Most of the research involving isotopes had been theoretical, relating to determining atomic structures and studying the then-mysterious properties of radioactivity. The Manhattan Project changed all that. The dire urgency of the war effort led to the development of sophisticated techniques for separating and identifying isotopes. One of these techniques, Isotope Ratio Mass Spectrometry (or IRMS for short), which is used to measure the relative abundance of isotopes in a sample, is now an important tool for studying and using isotopes.

Furthermore, the stable isotopic composition of matter has been recognized since about 1945 as a criterion for highly-specifically differentiating one material from another with the same elemental composition. In the field of geochemical oil exploration and prospecting, measurement of the isotopic compositions of large numbers of individual organic compounds of oil samples from various oil reservoirs have assisted in clarifying the origin of specific compounds correlating the organic compounds with particular petroleum sources, recognizing the existence of multiple petroleum sources, examining the mechanisms of petroleum generation, source mixing, and improving the sensitivity of petroleum migration studies. This information, particularly in connection with seismological data, can be used to predict locations of other oil reservoirs to which oil may have migrated from a common source of generation or formation.

Isotope ratio monitoring has had further applications in the biomedical field, wherein non-radioactive and stable isotopes and radioactive isotopes are used as tracer labels in drug metabolism and other biomedical studies where natural variations in isotopic abundances may also carry additional information regarding sources and fates of metabolites. Additionally, radioactive and stable isotopic labeling apparatus and methods in the medical fields employ typically costly labeled compounds having isotope ratios much different than those found in their natural abundance.

Despite the technologies available for labeling materials, such as, for example drug and food products, most rely on extrinsically doping, or tagging the bulk products. A bulk product can be is formulation containing one or more active ingredients with one or more excipients. Alternatively, a bulk product can be the active ingredient itself, as for example the active pharmaceutical ingredient (API) of a drug formulation. Typically, such bulk materials have been doped by extrinsically adding a taggant to the bulk substance. For example it has been reported to add polypeptides into a substance as a means for identifying the bulk substance. In other cases, bulk materials are tagged by adding nucleic acids of various nucleotide lengths. In other cases, DNA strands are used as a taggant in conjunction with PCR (polymerase chain reaction) for authenticating an object. In yet other cases, polymeric materials such as nucleic acid polymer particles are extrinsically added to the material.

Chemical substances have also been tagged with various isotopes, whether radioactive or non-radioactive. However, this isotopic tagging involves separately incorporating or adding the desired isotope at a desired position into the chemical substance—in other words making one or more position-specific substitutions. As a non smiting example, consider acetic acid where one of the hydrogen atoms on the methyl group of the acetic acid has been replaced with a deuterium atom.

However, these extrinsic and positional isotopic compositions and their related authentication and identification methods have the disadvantage of either chemically modifying a substance to incorporate an isotope at a specific position or of adding or mixing in another material into the desired substance as a dopant. These methods can have the undesired effects of changing, modifying, or contaminating the substance, which could be particularly unacceptable from a performance, safety, or regulatory standpoint, as for example for a food or drug product or for a fine chemical or high performance substance. Also, some of these methods either require radioactive isotopes or require the generation of radioactive isotopes, which could be impractical, if not unacceptable in certain instances.

Furthermore, the analytical methods needed to evaluate extrinsically doped bulk substances or positionally modified compounds can be impractical. Bulk materials, whether a formulation or an active ingredient, that have been extrinsically doped would need to be evaluated by bulk stable isotopic analysis (also known as BSIA). Positionally modified compounds would need to be evaluated by position specific isotopic analysis (also known as PSIA). A further analytical method known as compound specific isotopic analysis (also known as CSIA) would not, however, be useful for evaluating either extrinsically doped bulk substances or positionally modified compounds, because CSIA would instead be applicable to evaluating the statistical isotopic distribution of the compound as a whole.

See, U.S. Pat. No., 8,864,038 B2, to Marka et al., issued Oct. 21, 2014; U.S. Pat. No. 5,451,505, to Dollinger, issued Sep. 19, 1995; U.S. Pat. No. 4,359,353, to Kydd, issued Nov. 16, 1982; U.S. Patent Application Publication No. US 2014/0220576 A1, to Macula, published Aug. 7, 2014; and U.S. Patent Application Publication No. US 2010/0255465 A1, to Sleat, published Oct. 7, 2010; which are all incorporated by reference herein in their entirety.

Additionally, new processes and methods are needed for modifying or engineering the isotopic composition of materials. The reason for this is that current methods have instead been developed to dope, tag, or positionally label substances, and are not applicable to the actual design or engineering of substances with a statistically defined isotopic composition.

Therefore, new methods, compositions, and systems are needed for identifying, authenticating, and marking substances. It is apparent from the above there is an ongoing need for such new methods, compositions, and systems for selectively, reproducibly, and non-extrinsically and non-positionally engineering the isotopic composition of a wide variety of chemical and biological compounds.

### BRIEF DESCRIPTION OF THE FIGURES

FIG. 1 depicts the isotopic composition of a reaction product plotted as a function of reaction yield. The isotopic composition, δ, increases as the reaction yield approaches 1, i.e. as the reaction approaches completion. The symbol, ‰, designates permil or what is also referred to as parts per thousand. The reactant or reactants, the process, and the product or products are indicated on the plot.

FIG. 2 depicts the carbon-isotopic composition (δ ¹³C) of synthetic intermediates (C, E, G) and the final product, I, as a function of reaction step with (upper line) and without (lower line) the contributions of partial-reaction completion (f) and isotopic fractionation (ε).

FIG. 3 depicts the carbon-isotopic differences between isotopic compositions predicted and those which would be observed in the absence of isotope effects, δₚ*-δₚ, ‰, corresponding to the values in Table 1 as presented in the patent application.

FIG. 4 depicts a system for continuously monitoring the progress of a chemical or a biological process, in which a gaseous product (or by-product, e.g., CO₂) is generated. This system illustrates a stirred reactor vessel and a sampling device, which in this case is a carrier gas line for blowing a carrier gas through or over the chemical or biological process to continuously sample the chemical or biological process or to collect the gaseous product or by-product. The system also depicts an effluent tube, which is a part of the sampling device, which feeds in to an isotope analyzer and an associated computerized data system (CDS). The interface is essentially the connection between the sampling device (in this case the effluent tube) and the isotope analyzer.

FIGS. 5A and 5B depict isotopic compositions for ¹³C and ¹⁸O of the starting reactant ether compound, 2-bromo-6-methoxynaphthalene, compound 13, used in the synthesis of naproxen as per Example 6. The data is presented as counts (y-axis) versus isotopic enrichment (x-axis). The δ ¹³C data is reported in FIG. 5A as ‰ versus VPDB (Vienna Peedee Belemnite) on the x-axis. The δ ¹⁸O data is reported in FIG. 5B as ‰ versus VSMOW (Vienna Standard Mean Ocean Water) on the x-axis.

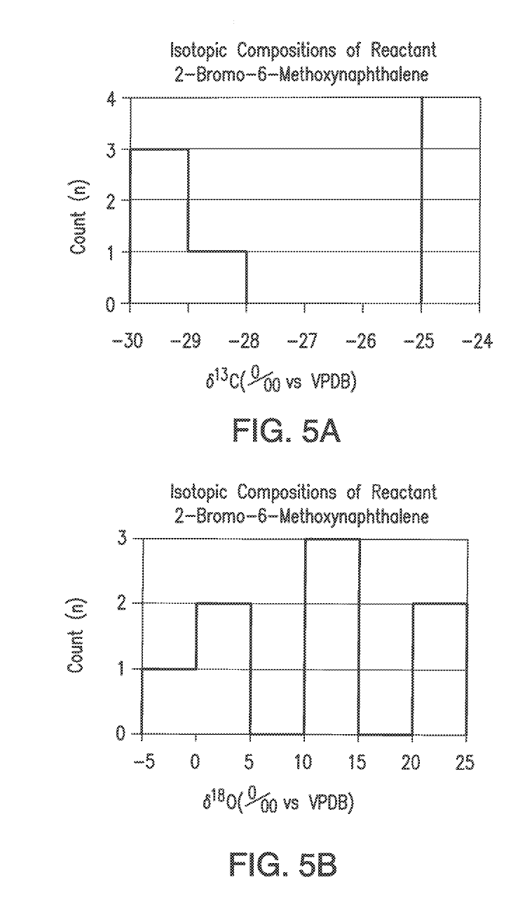

FIG. 6 depicts the ¹³C starting reactant isotopic composition of the starting reactant ether compound, 2-bromo-6-methoxynaphthalene, compound 13, for the preparation of naproxen as per Example 6. Three different sources [Sample Sources 1, 2, and 3 (Alfa Aesar, Combi-Blocks, and Matrix, respectively); each Sample Source represents 3 individual samples or n=3] for compound 13 are used (left side of figure) to produce the corresponding (±)-naproxen of indicated isotopic composition (right side of figure). The predicted IMB (isotopic mass balance) is shown for each Sample Source (far right side of figure). The δ ¹³C data is reported as ‰ versus VPDB (Vienna Peedee Belemnite) on the y-axis.

FIG. 7 depicts the ¹⁸O starting reactant isotopic composition of the starting reactant ether compound, 2-bromo-6-methoxynaphthalene, compound 13 for the preparation of naproxen as per Example 6. Three different sources [Sample Sources 1, 2, and 3 (Alfa Aesar, Combi-Blocks, and Matrix, respectively); each Sample Source represents 3 individual samples or n=3] for compound 13 are used (left side of figure) to produce the corresponding (±)-naproxen of indicated isotopic composition (right side of figure). The predicted IMB (isotopic mass balance) is shown for each Sample Source (far right side of the figure). The δ ¹⁸O data is reported as ‰ versus VSMOW (Vienna Standard Mean Ocean Water) on the y-axis.

FIG. 8 depicts the D (²H) starting reactant isotopic composition of the starting reactant ether compound, 2-bromo-6-methoxynaphthalene, compound 13 for the preparation of naproxen as per Example 6. Three different sources [Sample Sources 1, 2, and 3 (Alfa Aesar, Combi-Blocks, and Matrix, respectively), and 3; each Sample Source represents 3 individual samples or n=3] for compound 13 are used (left side of figure) to produce the corresponding (±)-naproxen of indicated isotopic composition (right side of figure). The predicted IMB (isotopic mass balance) is shown for each Sample Source (far right side of the figure). The δ D data is reported as ‰ versus VSMOW (Vienna Standard Mean Ocean Water) on the y-axis.

FIG. 9 depicts the observed versus predicted carbon isotopic compositions of racemic (±) naproxen values demonstrating (±1 standard deviation) correspondence between mass-balance/isotope-mass balance estimation and observed values

FIG. 10 depicts the observed versus predicted oxygen isotopic compositions of racemic (±) naproxen values demonstrating (±2 standard deviations) correspondence between mass-balance/isotope-mass balance estimation plus H₂O/naproxen equilibration and observed values.

FIG. 11 depicts the observed versus predicted hydrogen (deuterium) isotopic compositions of racemic (±) naproxen values showing the relationship between mass-balance/isotope-mass balance estimation plus H₂O/naproxen equilibration and observed values.

FIG. 12 depicts the superposition of the naproxen samples produced for this study of Example 6 [Sample Sources 1, 2, and 3 (Alfa Aesar, Combi-Blocks, and Matrix, respectively] over the naproxen isotope values previously observed in a cooperative study with the US FDA, Division of Pharmaceutical Analysis (DPA) showing that unknown naproxen samples could be correctly identified with the technology of the present invention according to manufacturer and/or country of origin (India, Manufacturer A; India, Manufacturer B; Italy, Manufacturer, C; Italy, Manufacturer D; Ireland, Manufacturer E; and USA, Manufacturer F). See, Wokovich, A. M., J. A. Spencer, B. J. Westenberger, Buhse, and J. P. Jasper. (2005) Stable isotopic composition of the active pharmaceutical ingredient (API) Naproxen, J. Pharm. Biomed. Anal., 38:781-784).

### SUMMARY OF THE INVENTION

The present invention relates to molecular isotopic engineering. The present invention relates to a method or process for preparing a target compound of a statistically defined isotopic composition comprising the step of reacting one or more reactant compounds, wherein each reactant compound is of a statistically defined isotopic composition. The reactant compound is reacted in a chemical process or a biological process thereby generating an isotopic mass balance, or further, an isotopic fractionation to produce the target compound. The present invention also relates to a statistically defined isotopic composition of a target compound. The statistically defined isotopic composition comprises an internal marker, and can be used as, for example, a security feature, an identity indicator, or a purity indicator of the target compound.

### DETAILED DESCRIPTION OF THE INVENTION

The present invention relates to method for preparing a target compound of a statistically defined isotopic composition comprising the step of reacting one or more reactant compounds, wherein each reactant compound is of a statistically defined isotopic composition, in a chemical process or a biological process generating an isotopic mass balance to produce the target compound. In alternative embodiments having two or more reactants compounds, at least one of the reactant compounds is of a statistically defined isotopic composition.

In one aspect the present invention relates to a method wherein the reactant compound comprises one or more isotope ratios from elements present in the reactant compound and the target compound comprises one or more isotope ratios from elements present in the target compound.

In another aspect the present invention relates to a method for preparing a target compound of a statistically defined isotopic composition comprising the step of reacting a first reactant compound of a statistically defined isotopic composition with a second reactant compound of a statistically defined isotopic composition in a chemical process or a biological process generating an isotopic mass balance to produce the target compound.

In another aspect the present invention relates to a method wherein the first reactant compound comprises one or more isotope ratios from elements present in the first reactant compound, the second reactant compound comprises one or more isotope ratios from elements present in the second reactant compound, and the target compound comprises one or more isotope ratios from elements present in the target compound.

In another aspect the present invention relates to method wherein the chemical process or the biological process further generates an isotopic fractionation.

In another aspect the present invention relates to a method wherein the elements are selected from elements that have two or more isotopes.

In another aspect the present invention relates to a method wherein the elements are selected from hydrogen, carbon, nitrogen, oxygen, sulfur, chlorine, bromine, and combinations thereof.

In another aspect the present invention relates to a method wherein the isotopes are stable isotopes.

In another aspect the present invention relates to a method where the stable isotopes are selected from ¹H, ²H, ¹²C, ¹³C, ¹⁴N, ¹⁵N, ¹⁶O, ¹⁸O, ³²S, ³⁴S, ³⁵Cl, ³⁷Cl, ⁷⁹Br, and ⁸¹Br and combinations thereof.

In another aspect the present invention relates to a method wherein the isotope ratios are selected from the following, pairs of isotopes: ¹H and ²H, ¹²C and ¹³C, ¹⁴N and ¹⁵N, ¹⁶O and ¹⁸O, ³²S and ³⁴S, ³⁵Cl and ³⁷Cl, and ⁷⁹Br, and ⁸¹Br.

In another aspect the present invention relates to a method wherein the isotope ratios are selected from the following isotope ratios ²H/¹H, ¹³C/¹²C, ¹⁵N/¹⁴N, ¹⁸O/¹⁶O, ³⁴S/³²S, ³⁷Cl/³⁵Cl, and ⁸¹Br/⁷⁹Br.

In another aspect the present invention relates to a method wherein the isotope ratio is ²H/¹H.

In another aspect the present invention relates to a method wherein the isotope ratio is ¹³C/¹²C.

In another aspect the present invention relates to a method wherein the isotope ratio is ¹⁵N/¹⁴N.

In another aspect the present invention relates to a method wherein the isotope ratio is ¹⁸O/¹⁶O.

In another aspect the present invention relates to a method wherein the isotope ratio is ³⁴S/³²S.

In another aspect the present invention relates to a method wherein the isotope ratio is ³⁷Cl/³⁵Cl.

In another aspect the present invention relates to a method wherein the isotope ratio is ⁸¹Br/⁷⁹Br.

In another aspect the present invention relates to a method wherein the chemical process or the biological process is a catalyzed process.

In another aspect the present invention relates to a method wherein the catalyzed process is an enzymatically catalyzed process.

In another aspect the present invention relates to a method wherein the chemical process or the biological process is a chemical process.

In another aspect the present invention relates to a method wherein the chemical process is a chemical reaction.

In another aspect the present invention relates to a method wherein the chemical reaction is a batch chemical reaction.

In another aspect the present invention relates to a method wherein the chemical reaction is a continuous chemical reaction.

In another aspect the present invention relates to a method wherein the continuous chemical reaction is a flow chemical reaction.

In another aspect the present invention relates to a method wherein the target compound is a pharmaceutical product.

In another aspect the present invention relates to a method wherein the chemical process or the biological process is a biological process.

In another aspect the present invention relates to a method wherein the biological process is a biological reaction.

In another aspect the present invention relates to a method wherein the biological reaction is a batch biological reaction.

In another aspect the present invention relates to a method wherein the biological reaction is a continuous biological reaction.

In another aspect the present invention relates to a method wherein the continuous biological reaction is a flow biological reaction.

In another aspect the present invention relates to a method wherein the target compound is a biological product.

In another aspect the present invention relates to a method wherein the statistically defined isotopic composition of the target compound is an internal marker.

In another aspect the present invention relates to a method wherein the statistically defined isotopic composition of the target compound is a security feature.

In another aspect the present invention relates to a method wherein the statistically defined isotopic composition of the target compound is an identity indicator.

In another aspect the present invention relates to a method wherein the statistically defined isotopic composition of the target compound is a purity indicator.

In another aspect the present invention relates to a statistically defined isotopic composition of a target compound prepared by a method comprising the step of reacting one or more reactant compounds, wherein each reactant compound is of a statistically defined isotopic composition, in a chemical process or a biological process generating an isotopic mass balance to produce the statistically defined isotopic composition in the target compound. In alternative embodiments having two or more reactants compounds, at least one of the reactant compounds is of a statistically defined isotopic composition.

In another aspect the present invention relates to a statistically defined isotopic composition of a target compound wherein the reactant compound comprises one or more isotope ratios from elements present in the reactant compound and the target compound comprises one or more isotope ratios from elements present in the target compound.

In another aspect the present invention relates to a statistically defined isotopic composition of a target compound prepared by a method comprising the step of reacting a first reactant compound of a statistically defined isotopic composition with a second reactant compound of a statistically defined isotopic composition in a chemical process or a biological process generating an isotopic fractionation to produce the statistically defined isotopic composition in the target compound.

In another aspect the present invention relates to a statistically defined isotopic composition of a target compound wherein the first reactant compound comprises one or more isotope ratios from elements present in the first reactant compound, the second reactant compound comprises one or more isotope ratios from elements present in the second reactant compound, and the target compound comprises one or more isotope ratios from elements present in the target compound.

In another aspect the present invention relates to a statistically defined isotopic composition of a target compound wherein the chemical process or the biological process further generates an isotopic fractionation.

In another aspect the present invention relates to a statistically defined isotopic composition wherein the elements are selected from elements that have two or more isotopes.

In another aspect the present invention relates to a statistically defined isotopic composition wherein the elements are selected from hydrogen, carbon, nitrogen, oxygen, sulfur, chlorine, bromine, and combinations thereof.

In another aspect the present invention relates to a statistically defined isotopic composition wherein the isotopes are stable isotopes.

In another aspect the present invention relates to a statistically defined isotopic composition where the stable isotopes are selected from ¹H, ²H, ¹²C, ¹³C, ¹⁴N, ¹⁵N, ¹⁶O, ¹⁸O, ³²S, ³⁴S, ³⁵Cl, ³⁷Cl, ⁷⁹Br, and ⁸¹Br and combinations thereof.

In another aspect the present invention relates to a statistically defined isotopic composition wherein the isotope ratios are selected from the following pairs of isotopes: ¹H and ²H, ¹²C and ¹³C, ¹⁴N and ¹⁵N, ¹⁶O and ¹⁸O, ³²S and ³⁴S, ³⁵Cl and ³⁷Cl, and ⁷⁹Br, and ⁸¹Br.

In another aspect the present invention relates to a statistically defined isotopic composition wherein the isotope ratios are selected from the following isotope ratios: ²H/¹H, ¹³C/¹²C, ¹⁵N/¹⁴N, ¹⁸O/¹⁶O, ³⁴S/¹⁶S, ³⁷Cl/³⁵Cl, and ⁸¹Br/⁷⁹Br.

In another aspect the present invention relates to a statistically defined isotopic composition wherein the isotope ratio is ²H/¹H.

In another aspect the present invention relates to a statistically defined isotopic composition wherein the isotope ratio is ¹³C/¹²C.

In another aspect the present invention relates to a statistically defined isotopic composition wherein the isotope ratio is ¹⁵N/¹⁴N.

In another aspect the present invention relates to a statistically defined isotopic composition wherein the isotope ratio is ¹⁸O/¹⁶O.

In another aspect the present invention relates to a statistically defined isotopic composition wherein the isotope ratio is ³⁴S/³²S.

In another aspect the present invention relates to a statistically defined isotopic composition wherein the isotope ratio is ³⁷Cl/³⁵Cl.

In another aspect the present invention relates to a statistically defined isotopic composition wherein the isotope ratio is ⁸¹Br/⁷⁹Br.

In another aspect the present invention relates to a statistically defined isotopic composition wherein the chemical process or the biological process is a catalyzed process.

In another aspect the present invention relates to a statistically defined isotopic composition wherein the catalyzed process is an enzymatically catalyzed process.

In another aspect the present invention relates to a statistically defined isotopic composition wherein the chemical process or the biological process is a chemical process.

In another aspect the present invention relates to a statistically defined isotopic composition wherein the chemical process is a chemical reaction.

In another aspect the present invention relates to a statistically defined isotopic composition wherein the chemical reaction is a batch chemical reaction.

In another aspect the present invention relates to a statistically defined isotopic composition wherein the chemical reaction is a continuous chemical reaction.

In another aspect the present invention relates to a statistically defined isotopic composition wherein the continuous chemical reaction is a flow chemical reaction.

In another aspect the present invention relates to a statistically defined isotopic composition wherein the target compound is a pharmaceutical product.

In another aspect the present invention relates to a statistically defined isotopic composition wherein the chemical process or the biological process is a biological process.

In another aspect the present invention relates to a statistically defined isotopic composition wherein the biological process is a biological reaction.

In another aspect the present invention relates to a statistically defined isotopic composition wherein the biological reaction is a batch biological reaction.

In another aspect the present invention relates to a statistically defined isotopic composition wherein the biological reaction is a continuous biological reaction.

In another aspect the present invention relates to a statistically defined isotopic composition wherein the continuous biological reaction is a flow biological reaction.

In another aspect the present invention relates to a statistically defined isotopic composition wherein the target compound a biological product.

In another aspect the present invention relates to a statistically defined isotopic composition wherein the statistically defined isotopic composition of the target compound is an internal marker.

In another aspect the present invention relates to a statistically defined isotopic composition wherein the statistically defined isotopic composition of the target compound is a security feature.

In another aspect the present invention relates to a statistically defined isotopic composition wherein the statistically defined isotopic composition of the target compound is an identity indicator.

In another aspect the present invention relates to a statistically defined isotopic composition wherein the statistically defined isotopic composition of the target compound is a purity indicator.

In another aspect the present invention relates to a method or statistically defined isotopic composition wherein the target compound is selected from a pharmaceutical, a biologic, a dietary supplement, a neutraceutical, a commodity chemical, or a fine chemical.

In another aspect the present invention relates to a method or statistically defined isotopic composition according wherein the pharmaceutical is selected from aripiprazole (Ability), esomeprazole (Nexium), adalimumab (Humira), rosuvastatin (Crestor), fluticasone, salmeterol, etanercept (Enbrel), duloxetine (Cymbalta), infliximab (Remicade), pegfilgrastim (Neulasta), sofosbuvir (Solvadi), glatiramer (Copaxone), insulin, heparin, rituximab (Rituxan), tiotroprium (Spiriva), sitagliptin (Januvia), efavirenz, emtricitabine, tenofovir, bevacizumab (Avastin), pregabalin (Lyrica), oxycodone (OxyContin), epoetin alfa (Epogen), celecoxib (Celebrex), valsartan (Diovan), imatinib(Gleevec), trastuzumab (Herceptin), ranibizumab (Lucentis), lisdexamfetamine (Vyvanse), ezetimibe (Zetia), and memantine (Namenba), naproxen, and pharmaceutically acceptable salts, esters, and prodrugs thereof.

In another aspect the present invention relates to sitagliptin, or a pharmaceutically acceptable salt or prodrug thereof having a statistically defined isotopic composition.

In another aspect the present invention relates to sitagliptin wherein the statistically defined isotopic composition has a δ¹³C statistical enrichment of −10.00‰ to +20.00‰ relative to a starting material.

In another aspect the present invention relates to sitagliptin wherein the starting material is

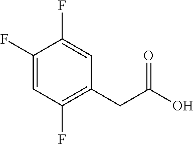 [SMILES: O=C(O)Cc1cc(F)c(F)cc1F]

or a pharmaceutically acceptable salt or ester thereof.

In another aspect the present invention relates to sitagliptin phosphoric acid salt monohydrate having a statistically defined isotopic composition.

In another aspect the present invention relates to naproxen, or a pharmaceutically acceptable salt or prodrug thereof having a statistically defined isotopic composition.

In another aspect the present invention relates to naproxen sodium salt having a statistically defined isotopic composition.

In another aspect the present invention relates to naproxen wherein the statistically defined isotopic composition has a δ¹³C statistical enrichment of −10.00‰ to +20.00‰ relative to a starting material.

In another aspect the present invention relates to naproxen wherein the starting material is selected from

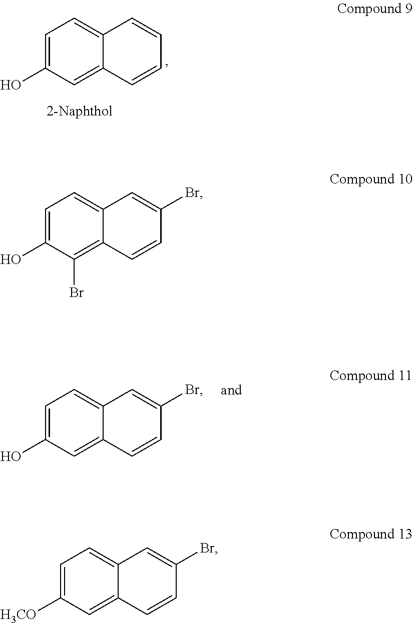 [SMILES: Oc1ccc2ccccc2c1 ; Oc1ccc2cc(Br)ccc2c1Br ; Oc1ccc2cc(Br)ccc2c1 ; "and" ; COc1ccc2cc(Br)ccc2c1]

and pharmaceutically acceptable salts or esters thereof, and combinations thereof.

In another aspect the present invention relates to naproxen wherein the starting material is

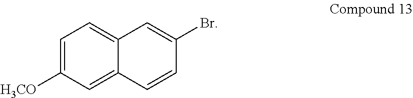 [SMILES: COc1ccc2cc(Br)ccc2c1]

In another aspect the present invention relates to (+)-(S)-Naproxen having a statistically defined isotopic composition.

In another aspect the present invention relates to (+)-(S)-Naproxen sodium salt having a statistically defined isotopic composition.

### Definitions

As used herein, the following terms have the following meanings unless expressly stated to the contrary:

The term “batch” as used herein refers to a process involving a quantity of material prepared or required for one operation or step, or the quantity produced at one operation. In a batch process, the output of that process can be passed to a subsequent process for further processing. A “batch process” or “batch processing” is in contrast to a “continuous process” or “continuous processing”.

The term “biological product” or “biologic product” as used herein refers to a biologically-produced medical product, which is commonly referred to as a “biologic”. Examples of biological products include medicinal products such as vaccines, blood, blood components, antibodies such as monoclonal antibodies, enzymes, proteins, and the like. Biological products also include materials for viral gene therapy for artificially manipulating a virus to include a desired piece of genetic material into a target gene or cell. In general, biological products are produced by biological processes, such as e.g., fermentation, cell cultures, extractions, purifications, and harvesting from biological sources. Biological products are also produced by genetic engineering techniques such as, e.g., recombinant DNA and RNA procedures, polymerase chain reaction (PCB) amplification, and the like. Biological products are also produced by derivatization and modification of natural product sources. Biological products generally are made by biological processes rather than chemical processes.

The term “continuous” as used herein refers to a “continuous process” and also a method or system for “continuously monitoring” a process, “continuously sampling a process”, and “continuously determining” (with respect to the process) whether it is a continuous process or a batch process. A “continuous process” is one that is designed to run non-stop. A “continuous process” or “continuous processing” is in contrast to a “batch process” or “batch processing”. “Continuously monitoring” means that the methods and systems are such that they sample, monitor, measure, or determine (i.e. make determinations with respect to the process) the processes of the present invention down to very small time intervals, such that the sampling, monitoring, measuring, or determining, for all intents and practical purposes, is essentially instantaneous. Such sampling, monitoring, measuring, or determining can then be conducted at one or more time points or at desired time intervals. Alternatively, the sampling, monitoring, measuring, or determining is made from a gaseous stream or outflow, or from a liquid stream or outflow from the chemical process or the biological process, a non-limiting example of such being wherein an inert gas, such as helium, is continuously run over or through the chemical or biological process to continuously sweep out or remove a gaseous product or by-product, such as CO₂ or CO. The isotopic information is continuously determined on the gaseous product or by-product to monitor the progress of the chemical process or the biological process. In the foregoing described in this paragraph, the output of such sampling, monitoring, measuring, or determining is essentially continuous.

The term “first time point” as used herein refers to the first point in time or the initial point in time at which the process is sampled and the first or initial isotope information is determined or assessed. The term “first time point” is synonymous with “initial time point”. The term “first time point” or “initial time point” is intended to be distinguished from one or more subsequent time points or later time points, at which the process is sampled and subsequent or later isotope information is determined or assessed.

The term “flow” as used herein refers to a continuous chemical or biological process wherein the feedstocks, starting materials or reactants are provided in a flow or stream and the desired product or products are removed as an effluent flow or stream.

The term “incremental” as used herein refers to an additional increase in quantity, and in most cases a small or minute, but measureable, increase in quantity. The term as used herein refers to an incremental yield for a chemical or biological process.

The term “instantaneous” as used herein refers to something that happens or occurs very quickly or in an instant, or in other words, in a very small, but measureable increment of time. The term “instantaneous” as used herein also refers to an instantaneous yield for a chemical or biological process. Because it is recognized that the methods and systems of the present invention may not strictly provide instantaneous sampling, monitoring, or measuring, the term “instantaneous” is also meant to include the terns “substantially instantaneous” and “essentially instantaneous”, to convey the concept that for all intents and practical purposes these methods and systems are instantaneous.

The term “molecular isotopic engineering” or “MIE” as used herein refers to a manufacturing method by which the stable-isotopic composition of chemical or biological products can be prepared, or engineered, by selection of reagents (i.e., starting materials and synthetic intermediates) of known isotopic composition and of a given synthetic pathway. Thus, manufacturing under specified conditions products will generate products with a statistically-delimited isotopic range. Purposeful variations of the isotopic compositions of the starting material and/or of the isotopically-fractionating reaction conditions (e.g., reaction rate via temperature, pressure, time, reagent concentration, etc.) can be used to substantially predetermine the isotopic ranges for given product batches. This isotopic fractionation can be made to occur further beyond the isotopic mass balance that would normally occur in a chemical or biological process

The terms “(±)-naproxen” and “(R,S)-naproxen” can be used interchangeable and are both used in their conventional sense to indicate a racemic mixture of the two enantiomers of naproxen. R-naproxen is known to be the (−) enantiomer, because it rotates plane polarized light in the levorotatory, counterclockwise, or left hand direction. S-naproxen is known to be the (+) enantiomer, because it rotates plane polarized light in the dextrorotary, clockwise, or right hand direction.

The term “pharmaceutically acceptable” as used herein refer to derivatives of the disclosed compounds wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, ethane sulfonic, fumaric, glucoheptonic, gluconic, glutamic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sutfanilic, sulfuric, tannic, tartaric, and toluene sulfonic. The pharmaceutically acceptable salts of the present invention can be synthesized from a parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 18th ed. (Mack Publishing Company, 1990).

The term “prodrug” as used herein is intended to include any covalently bonded carriers that release an active parent drug of the present invention in vivo when such prodrug is administered to a mammalian subject. Prodrugs the present invention are prepared by modifying functional groups present in the compound in such a way that the modifications are cleaved, either in routine manipulation or in vivo, to the parent compound. Prodrugs include compounds of the present invention wherein for example, a hydroxy, amino, carboxylic acid, or sulfhydryl group is bonded to any group that, when the prodrug of the present invention is administered to a mammalian subject, cleaves to form a free hydroxyl, free amino, free carboxylic acid, or free sulfhydryl group, respectively. Examples of prodrugs include, but are not limited to, acetate, formate, and benzoate derivatives of alcohol and amine functional groups in the compounds of the present invention. Since prodrugs are known to enhance numerous desirable qualities of pharmaceuticals (e.g., solubility, bioavailability, manufacturing, etc.) the compounds of the present invention may be delivered in prodrug form. Thus, the present invention is intended to cover prodrugs of the presently claimed compounds, methods of delivering the same and compositions containing the same.

The term “process” as used herein refers to one or more actions or operations for making, producing or manufacturing a product. The term is intended to include chemical processes and biological processes. The term process is also is also intended to include the sum of one or more reactions, which can be chemical reactions or biological reactions. The processes and reactions include feedstocks, starting materials, reactants, solvents, catalysts; physical parameters such as temperature, pressure, agitation, atmospheric conditions, aeration, and gas through-put; and time variables; and the like.

The terms “reaction or reactions” as used herein refer to the chemical or biological reactions of the processes of the present invention. A reaction is generally a discrete chemical or biological step or transformation.

The term “stable isotope” or “stable isotopes” as used herein refers to those isotopes that have never been observed to decay. It is recognized that all isotopes will eventually decay. Some isotopes such as hydrogen-7 (⁷H) and lithium-4 (⁴Li) have half-lives on the order of 10⁻²⁴ seconds, whereas, in contrast, calcium-48 (⁴⁵Ca) and tellurium-148 (¹⁴⁸Te) have half-lives on the order of 10²⁴ years. The stable isotopes useful in the present invention are generally the naturally-occurring stable isotopes of hydrogen, carbon, nitrogen, oxygen, sulfur, chlorine, and bromine. More specifically these naturally-occurring stable isotopes are hydrogen (hydrogen-1 or ¹H), deuterium (hydrogen-2 or ²H), carbon-12 (¹²C), carbon 13 (¹³C), nitrogen-14 (¹⁴N), nitrogen-15 (¹⁵N), oxygen-16 (¹⁶O), oxygen-18 (¹⁸O), sulfur-32 (³²S), sulfur-34 (³⁴S), chlorine-35 (³⁵Cl), chlorine-37 (³⁷Cl), bromine-79 (⁷⁹Br), and bromine-81 (⁸¹Br).

The term “stream” as used herein refers to a continuous flow process wherein either the continuous feedstock, starting materials, or reactants are conveyed to or flow to the process or the reactor or vessel in which the process occurs; or wherein the effluent containing the desired product or products is removed from or where conveyed from, or flows from the process or the reactor or vessel in which the process occurs. The process can be considered a flow or stream process.

The abbreviation “VPDB”, as used herein, stands for Vienna Peedee Belemnite, which is a reference standard for the stable isotopes of carbon. The original peedee belemnite sample was from fossilized shells of an extinct organism called a belemnite, which was collected from the banks of the Pee Dee River in South Carolina.

The abbreviation “VSMOW”, as used herein, stands for Vienna Standard Mean Ocean Water, which is a reference water standard defining the isotopic composition of fresh water, and which is used as a reference standard for the stable isotopes of hydrogen and oxygen.

The symbol, δ, is a measure of isotopic abundance, and δ is usually reported as the difference in parts per thousand, or paring (‰), from an international standard. The symbol, δ, can be negative or positive depending on whether the sample is enriched or depleted in the heavy isotope relative to a standard.

The symbol, ‰, designates peril or what is also referred to as parts per thousand,

Molecular Isotopic Engineering

As discussed above, molecular isotopic engineering or “MIE” is the manufacturing method described herein by which the stable-isotopic composition of chemical or biological products can be prepared, or engineered, by selection of reagents (i.e., starting materials and synthetic intermediates) of known isotopic composition and of a given synthetic pathway. Thus, manufacturing under specified conditions products will generate products with a statistically-delimited isotopic range. Purposeful variations of the isotopic compositions of the starting material and/or of the isotopically-fractionating reaction conditions (e.g., reaction rate via temperature, pressure, time, reagent concentration, etc.) can be used to predetermine the isotopic ranges given product batches. This isotopic fractionation can be made to occur further beyond the isotopic mass balance that would normally occur in a chemical or biological process.

Consider the situation where molecule “A” is reacted with molecule “B” to produce molecule “C”.

A+B→C

wherein A±σ_{A}, represents the standard deviation for the isotopic distribution for A,

B±σ_{B}, represents the standard deviation for the isotopic distribution for B,

C±σ_{C}, represents the standard deviation for the isotopic distribution for C, and

σ_{C}=[(σ_{A})²+(σ_{B})²]^{1/2}

(which gives the distribution of error for C, from A+B).

Also, consider that the isotopic provenance for a substance is a function of the starting reactants as well as the synthetic process by which the substance is made. In other words:

Isotopic Provenance=f(reactants, synthetic process).

As shown further below, equation 3 (isotope mass balance) is a default case of equations 4 and 5 (each of which expresses isotopic fractionations, Δ_{A}, Δ_{B}). That is, when the fractionations are zero, equations 4 and 5 default to equation 3 (i.e., isotope mass balance). See, J. P. Jasper, L. E. Wearier, and J. M. Hayes, Process Patent Protection: Characterizing Synthetic Pathways by Stable-Isotope Measurements, Pharmaceutical Technology, 2007, 31(3):68-73, which is incorporated by reference herein in its entirety.

Stable Isotope Identification Methods

Methods and systems for isotope identification are described in U.S. Pat. No. 7,323,341 B1, to Jasper, issued Jan. 29, 2008 and U.S. Pat. No. 8,367,414 B2, to Jasper, issued Feb. 5, 2013, which are incorporated by reference herein in their entirety. U.S. Pat. No. 7,323,341 describes a stable isotopic identification and method for identifying products using naturally occurring isotopic concentrations or isotopic ratios in products, especially in the pharmaceutical industry, and more particularly to an identification and a method utilizing such isotopic concentrations or ratios in a machine readable form for identifying products and tracking products through manufacturing, marketing and use of a product, and readily indexing product information to the product. U.S. Pat. No. 8,367,414 describes isotope analysis and, in particular, a field of analytical chemistry that is directed to the derivation of information regarding the origins of synthetic products from processes in which the amounts or ratios of isotopes in either synthetic starting materials, intermediates or products are traced.

Methods for characterizing manufacturing pathways using isotopic methods are also described. See, e.g., J. P. Jasper, L. E. Weaner, and J. M. Hayes, Process Patent Protection: Characterizing Synthetic Pathways by Stable-Isotope Measurements, Pharmaceutical Technology, 2007, 31(3):68-73, which is incorporated by reference herein in its entirety. This reference describes methods by which precise analyses of stable-isotopic abundances can be used in security and forensic applications for pharmaceutical materials. These methods include product and process authentication of raw materials, pharmaceutical intermediates, drug substances, formulated drug products, and synthetic pathways. Since the inception of these techniques, there have been yet further improvements in isotope ratio monitoring sensitivity and precision, as well as frequency, and a reduction in sample size requirements. See also, Jasper, J. P. (2004) Pharmaceutical security: Using stable isotopes to authenticate pharmaceutical materials. Tablets and Capsules 2(3):37-42; Jasper, J. P., R. C. Lyon, and L. E. Weaner (2005) Stable isotopes provide a new PAT [Process Analytical Technology] tool. Pharm. Mfg. 4(5):28-33; Wokovich, A. M., J. A. Spencer, B. J. Westenberger, L. F. Buhse, and J. P. Jasper. (2005) Stable isotopic composition of the active pharmaceutical ingredient (API) Naproxen. J. Pharm. Biomed. Anal., 38:781-784; Jasper, J. P., L. E. Weaner, and B. J. Duffy (2005) A preliminary multi-stable-isotopic evaluation of three synthetic pathways of Topiramate. J. Pharm. Biomed. Anal. 39:66-75; Jasper, J. P., L. E. Weaner, and B. J. Duffy (2004). A preliminary multi-stable isotopic evaluation of Topiramate. Forensic Isotope Ratio Mass Spectrometry (FIRMS) Newsletter 2(2):8-9; Jasper, J. P., B. J. Westenberger, J. A. Spencer, L. F. Buhse, and M. Nasr (2004) Stable isotopic characterization of active pharmaceutical ingredients. J. Pharm. Biomed. Anal. 35:21-30; Authentication News (2004). Two new FDA studies cover stable isotopic authentication. June 2004 10(5):5; and Jasper, J. P., J. S. Edwards, L. C. Ford and R. A. Corry. (2002). Putting the arsonist at the scene: “DNA” for the fire investigator? Gas chromatography/isotope ratio mass spectrometry. Fire Arson Investig. 51(2): 30-34; and Jasper, J. P. (2001). Quantitative estimates of precision for molecular isotopic measurements. Rap. Comm. Mass Spec. 15:1554-1557; which are all incorporated by reference herein in their entirety.

Measurements of the abundances of naturally occurring stable isotopes in pharmaceutical materials can be used to quantitatively characterize both the sources of the products and the synthetic processes used to produce them, as well as the progress of those processes. The methods and systems of the present invention utilize isotopic information for one or more isotope ratios from elements present in samples from the chemical or biological processes of interest.

For many products, e.g. such as a pharmaceutical product, the source of each atom is known in detail. For example, a methyl carbon will derive from a particular synthetic reactant, an amino nitrogen from another reactant, etc. The measured carbon or nitrogen isotopic composition of the final product will be the weighted average of all carbon or nitrogen positions within the molecule. In turn, this measured isotopic composition will be equal to the weighted average of the isotopic compositions at the precursor positions in the synthetic reactions as modified by generally only two factors: (i) if the synthetic reactions are non-quantitative, any isotope effects which modulate the transfer of material from reactant to products and (ii) in some cases, exchanges of isotopes between products and reaction media.

Isotopic calculations are based on two systems of equations. The first employs mass balances and the second involves integrated forms of rate equations that pertain to kinetically controlled isotopic fractionations. Equations describing mass balances are generally exact when cast in terms of fractional abundances [e.g., ¹³C/(¹²C+¹³C)]. In contrast, assessments of differential rates are based on isotope ratios (e.g., ¹³C/¹²C). When these systems are blended, either approximations or equations with multiple terms are employed. For details, see Hayes J M, 1004; ttp://www.nosmas.whoi.edu/docs/IsoCalcs.pdf, which is incorporated by reference herein in its entirety.

The relevant isotopic parameters are stoichiometry (n), isotopic abundance (δ), the magnitude of the isotopic effect (ε), and a variable related to conversion of reactants to products (f).

The symbol, n represents the stoichiometry of the reaction, more specifically the number of atoms of a given element (e.g., carbon) in a given molecule involved in the reaction.

As mentioned above, (δ), is a measure of isotopic abundance, and δ is usually reported as the difference in parts per thousand, or permil (‰), from an international standard. δ can be negative or positive depending on whether the sample is enriched or depleted in the heavy isotope relative to the standard. For example, in the case of carbon the difference is calculated as

δ¹³C(‰)=([Rₛₘₚₗ)/(R_{std})]−1)·(1000) (equation 1)

where Rₛₘₚₗ is the ¹³C/¹²C ratio of the sample and R_{std} is the ¹³C/¹²C ratio in the standard. δ is thus linearly proportional to the isotopic ratio in the sample. Standards are available from the International Atomic Energy Authority and a standard for each isotope is used to determine the zero point of an abundance scale for that isotope. Standards include a particular seawater sample for H and O, calcium carbonate for C, air for N, and a meteorite for S. When the sample is depleted in the heavy isotope relative to the standard, δ is negative and when the sample is enriched it has a positive value. If it has the same isotopic abundance then δ=0. See, J P Jasper, The Increasing Use of Stable Isotopes in the Pharmaceutical Industry, Pharm. Tech., 1999, 23(10):106-114, which is incorporated by reference herein in its entirety.

The magnitude of an isotope effect, (ε), is such that its value depends on details of the reaction and on the relative mass difference between isotopes. Effects are largest for D (deuterium) vs. H and smaller for heavier elements. In general, the values of ε are specific to individual positions within the molecules involved. The isotope effects are largest at the reaction site, much smaller at neighboring positions, and usually not measurable elsewhere. Like δ, ε relates to the isotopic difference between two materials (e. g., reactant and product) and is usually expressed in permil or parts per thousand. For example, for kinetic isotope effects, in the methods and systems employed here, ε=−10‰ means that a reaction site bearing the heavy isotope reacts 10 parts per thousand, or 1%, more slowly than a site bearing a light isotope. For equilibrium isotope effects, ε_{A/B}=15‰ would mean that, at equilibrium, A is enriched in the heavy isotope by 15 parts per thousand relative to B. Here, A and B refer to specific atomic positions that can be related by a chemical equilibrium.

f is a measure of the progress of a reaction. It is generally the most important variable governing fractionations caused by isotope effects. Its value ranges from 1 to 0 and depends on factors such as temperature, pressure, or availability of reactants. In equilibria (A⇄B), f indicates the position of the equilibrium, with f_{B}=1 indicating complete conversion to B and, at any position, f_{A}+f_{B}=1. In irreversible reactions, f_{X} indicates the portion of reactant X which remains unconsutned, with f_{X}→0 as the reaction proceeds to completion.

The precision of isotopic analyses is typically calculated by two methods. Pooled standard deviations of raw data are typically computed from sets of duplicate or triplicate measurements. From those pooled standard deviations, standard deviations of mean values pertaining to specific substances are calculated. More specifically, the standard deviation of a mean value is the pooled standard deviation divided by n^{1/2}, where n is the number of measurements performed on a given sample. See Jasper, J P, Quantitative estimates of precision for molecular isotopic measurements. Rap. Comm. Mass Spec., 2001 15:1554-1557, which is incorporated by reference herein in its entirety. For carbon, nitrogen, oxygen, and sulfur, the resulting 95% confidence intervals for a result are typically in the range of ±0.1- to ±0.4‰. For hydrogen, the 95% confidence interval is typically ±3‰.

Precise quantitation of stable isotopic compositions in pharmaceutical intermediates and products requires both mass balance and isotopic fractionation equations that are applicable to both single and multi-step reaction sequences. One starts from the most basic requirement of mass balance then considers isotopic fractionations in a single reaction.

Mass Balance

For A+B→C, where reactants A and B are quantitatively converted to product C, two mass balances can be written:

m_{A}+m_{B}=m_{C} equation (2)

m_{A}δ_{A}+m_{B}δ_{B}=m_{C}δ_{C} equation (3)

where, m_{A}, m_{B}, and m_{C} are molar amounts of carbon (or any other element) in A, B, and C and the isotopic compositions of that carbon (or any other element) in A, B, and C are given by δ_{A}, δ_{B}, and δ_{C}. Equation 2 is a mass balance (i.e., carbon in=carbon out) while equation 3 is an isotopic mass balance (¹³C in=¹³C out). Under the conditions postulated (quantitative conversion) the isotopic composition of C can be computed from those of A and B. See, Hayes J M, 1004; http://www.nosmas.whoi.edu/docs/IsoCalcs.pdf and J P Jasper, The Increasing Use of Stable Isotopes in the Pharmaceutical Industry, Pharm. Tech., 1999, 23(10):106-114, which are incorporated by reference herein in their entirety.

Isotopic Fractionation

For isotopic fractionations, calculations should take into account factors such as reaction completeness and isotope effects, as these will cause the isotopic composition of C to differ from that computed using the mass balance equation and assuming quantitative conversion of reactants to products. To provide a concrete example, assume that A is present in excess while B, the limiting reactant, is quantitatively converted to product. In that case

n_{A}(δ_{A}−Δ_{A})+n_{B}δ_{B}=n_{C}δ_{C} equation (4)

where n_{A}, n_{B}, and n_{C} represent the numbers of atoms of carbon (or any other element of interest) in A, B, and C. Because A is not quantitatively converted to product, the isotopic compositions of the A-derived positions in C can differ from those in the initial reactant. Here, that isotopic offset, or change, is expressed as Δ_{A}, where its value depends on the isotope effect(s) and on the fraction of A that remains unconsumed. If the reaction conditions, particularly the magnitude of the excess of A, are consistent, Δ_{A} will be constant. Because the n values are known exactly, Δ_{A} can be deter mined from equation 4 after isotopic analysis of the reactants and product (i.e., determination of δ_{A}, δ_{B}, and δ_{C}).

Values of δ_{A}, δ_{B}, and δ_{C} generally do not affect the values of Δ_{A}. Accordingly, once Δ_{A} is known for a given reaction and set of conditions, it is usually necessary only to know two of the δ values in order to compute the third. Thus, for example, when δ_{A}, δ_{A}, and δ_{B}, are known, the isotopic value of the product (δ_{C}) can be calculated.

If neither A nor B is completely consumed during the course of the reaction, and if the rate of the chemical reaction (or position of the chemical equilibrium) is sensitive to isotopic substitution on both reactants, it will be necessary to consider values of the offset, or change, of both Δ_{A} and Δ_{B}:

n_{A}(δ_{A}−Δ_{A})+n_{B}(δ_{B}−Δ_{B})=n_{C}δ_{C} equation (5)

If reaction conditions cannot be manipulated so that f_{A} and f_{B} (and thus Δ_{A} and Δ_{B}) can be independently driven to completion (i.e., zero), it will be possible to determine only the sum, n_{A} δ_{A}+n_{B}δ_{B}. From theoretical considerations, Δ_{A} and Δ_{B} can be evaluated separately for all values of f_{A} and f_{B} if the isotope effects are known. See, Scott, K M, Lu, X, Cavanaugh, C M, and Liu, J S, Geochim. Cosmochim. Acta, 2004; 68(3):433, which is incorporated by reference herein in its entirety.

Of course, isotopic fractionations like those discussed above accumulate during the different steps of a multi-step synthesis scheme. They can, however, be individually and systematically differentiated, not only for multiple reactants but also for multiple isotopes. To provide an example consider carbon-isotopic fractionations in a hypothetical four-step sequence:

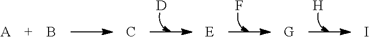

Illustrative carbon skeletons for reactants and products are shown below with pertinent quantities summarized in Table 1.

Illustrative Carbon Skeletons for Reactants and Products

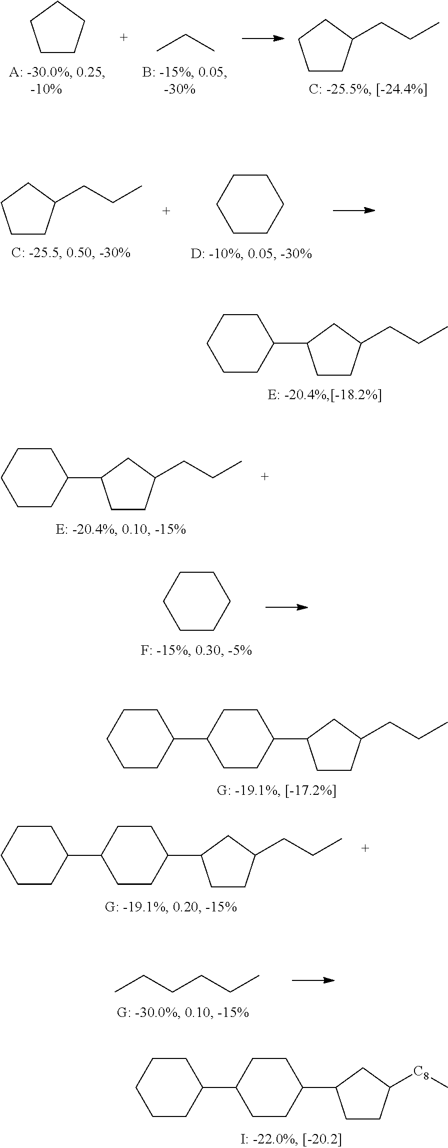

Carbon skeletons of reactants and products in a hypothetical four-step synthetic reaction scheme. This example illustrates the effects of the four key isotopic variables (n, δ, f, ε) on the isotopic compositions of the three synthetic intermediates (C, E, G) and of the final product, I (δ_{P}). For all eight reactants (left), the given numerical values are δ, f, and ε. For all four products (right), the numerical values are the isotopic compositions actually observed (δ_{P}) and that expected in the absence of isotope effects and incomplete consumption of reactants ([δ_{P}*]).

TABLE 1
Properties of Four-Step Synthetic Sequence^{a}

| Reactants | | | | | | Conditions | | | | Products | | |
|---|---|---|---|---|---|---|---|---|---|---|---|---|
| 1 | 2 | n₁ | n₂ | δ₁, ‰ | δ₂, ‰ | f₁ | f₂ | Σε₁, ‰ | Σε₂, ‰ | | δ_{P}*, ‰ | δ_{P}, ‰ |
| A | B | 5 | 3 | −30.0 | −15.0 | 0.255 | 0.055 | −10.0 | −30.0 | C | −24.4 | −25.5 |
| C | D | 8 | 6 | −25.5 | −10.0 | 0.50 | 0.05 | −30.0 | −5.0 | E | −18.2 | −20.4 |
| E | F | 14 | 6 | −20.4 | −15.0 | 0.10 | 0.30 | −15.0 | −5.0 | G | −17.2 | −19.1 |
| G | H | 20 | 6 | −19.1 | −30.0 | 0.20 | 0.10 | −15.0 | −15.0 | I | −20.2 | −22.0 |

^{a}The sequence of reactants and products is given by equation 6 in the text. The numbers of carbon atoms and the carbon-isotopic compositions of reactants 1 and 2 in each step are given by n₁, n₂, δ₁, and δ₂. The fractions of each reactant uncomsumed in each step are given by f₁ and f₂. The sums of all carbon isotope effects pertaining to each reactant are given by Σε₁ and Σε₂. The isotopic compositions that successive products would have in the absence of isotope effects are given by δ_{P}* and the isotopic compositions actually observed are given by δ_{P}.

The carbon numbers (n₁, n₂), initial isotopic compositions (δ₁, δ₂), fractions of reactants remaining unconsumed (f₁, f₂) and summed isotope effects (Σε₁, Σε₂) are chosen to be representative of a typical synthetic scheme. All isotope effects are assumed to be kinetic. Values of δ_{P}*, the isotopic compositions that would be observed if isotopic fractionations were absent, are calculated using equation 5 with Δ_{A}=Δ_{B}=0; that is, the simple mass balance equations 2-3. Values of δ_{P}, the isotopic compositions that would actually be observed for the successive products, are calculated using exact forms of integrated rate equations. See Scott, K M, Lu, X, Cavanaugh, C M, and Liu, J S, Geochim. Connochim. Acta, 2004; 68(3):433, which is incorporated by reference herein in its entirety.

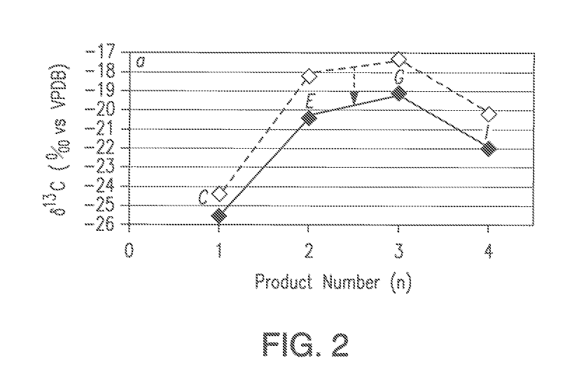

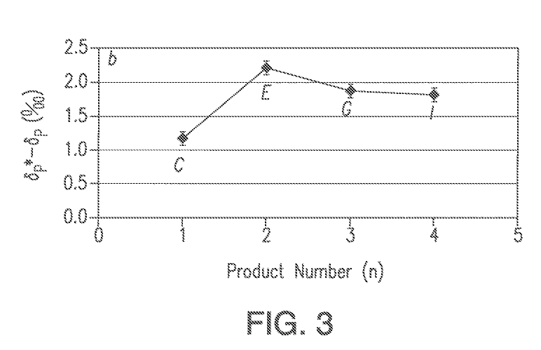

The foregoing illustrates the interplay of the four factors that control the isotopic compositions of manufactured products, namely the stoichiometries and isotopic compositions of the starting materials, isotope effects associated with the synthetic reactions, and the deuce to which conversions of precursors to products are quantitative. The isotopic compositions of products are generally dominated by the initial isotopic abundance of the precursor materials and are variously modulated (viz., depleted) by the degree of completion (f) and the magnitude of any isotopic effects (ε, FIG. 2). A plot that summarizes the difference between the isotopic compositions that are predicted and those that would be observed in the absence of isotope effects (δ_{P}*−δ_{P}*) is shown in FIG. 3. These values are also shown in the last two columns of Table 1. In the first synthetic step, isotope effects on reactant B are rather large, but that reactant is consumed almost completely. The resulting isotopic fractionation is less than 1‰ (the larger value shown in FIG. 3 pertains to the product and reflects fractions affecting both reactants). In the second step, a large isotope effect and poor conversion of reactant C lead to a large isotopic fractionation at the reaction site. However, fractionation is diluted now that the product contains 14 carbon atoms. As shown in FIG. 3, the overall difference between real and hypothetical unfractionated products is barely doubled. In the remaining steps, where isotope effects are moderate and consumption of reactants is relatively efficient, isotopic generally fractionation declines.

For a chemical or biological process, one can monitor the equilibrium between two isotopes, which are designated as “A” and “B”. Consider the case of a general system in which there are three different atoms or isotopes under consideration:

A+B→P

where A, B, and P contain n_{A}, n_{B}, and n_{P} atoms of the element under consideration. The system can be described by the following equation for determining the progress of the process via the isotopic abundance, δ:

$${δ}_{P} = {δ}_{P}^{*} - \frac{1}{{n}_{P}} [ \frac{{f}_{A}}{1 - {f}_{A}} ln {f}_{A} ( {ɛ}_{A 1} + {ɛ}_{A 2} + … ) + \frac{{f}_{B}}{1 - {f}_{B}} ln {f}_{B} ( {ɛ}_{B 1} + {ɛ}_{B 2} + … ) ] \qquad equation ( 6 )$$

In equation 6, δ_{P} and δ_{P}* are the observed isotopic composition of the product and the idealized isotopic composition of the product, respectively, ε_{A1}, ε_{B2}, etc. are the primary kinetic isotope effects at the reaction sites in A and B, respectively, and ε_{A2}, ε_{B2}, etc. are the secondary isotope effects, and f is a measure of the progress of the reaction.

Isotope Analyzer

The systems of the present invention comprise an isotope analyzer.

In theory, a wide range of isotope analyzers can be used in the systems of the present invention. The isotope analyzer is a device for measuring or determining the desired stable isotope ratios of the sampled process. Examples of isotope analyzers useful for the methods and systems of the present invention include those selected from: (a) cavity ring-down spectrometer (CRDS), (b) an isotope ratio mass spectrometer (IRMS), and (c) a nuclear magnetic resonance (nmr) spectrometer.

Cavity Ring-Down Spectrometer (CRDS)

Cavity ring-down spectroscopy (CRDS) is an optical spectroscopic technique utilizing a cavity ring-down spectrometer (CRDS). The method is highly sensitive, down to the 0.1% level, and is used to measure the light absorption of samples, i.e, the absolute optical extinction, that scatter and absorb light such as gas samples. A common cavity ring-down spectrometer configuration comprises a laser used to illuminate a high-finesse optical cavity, which essentially comprises two highly reflective mirrors. When the laser is in resonance with a cavity mode, the intensity of the laser light builds up in the cavity due to constructive interference. When the laser is turned off, the exponentially decaying light intensity leaking from the cavity is measured. This decaying laser light is reflected between the mirrors many thousands of time giving an effective path length on the order of kilometers.

When a sample is placed in the cavity, such as a sample containing a desired isotope, the intensity of the light decreases faster due to the absorption of the sample. The cavity ring-down spectrometer measures how long it takes for the light to decay, or “ring-down” to 1/e of its initial intensity both with and without the sample, thus giving a measure of the amount of the sample absorbing the laser light. See, Giel Berden; Rudy Peeters; Gerard Meijer (2000). “Cavity ring-down spectroscopy: Experimental schemes and applications”. International Reviews in Physical Chemistry 19 (4): 565-607; and Paldus, B. A. and Kachanov, A. A., An Historical Overview of Cavity Enhanced Methods (Einstein Centennial Review Article), Canadian Journal of Physics, 83, pp. 975-999 2005 NRC; which are incorporated by reference herein in their entirety.

An example of a cavity ring-down spectrometer useful in the methods and systems of the present invention includes a Picarro CRDS G2131-i Analyzer sold by Picarro Inc., 3105 Patrick Henry Drive, Santa Clara, Calif. 95054.

Isotope Ratio Mass Spectrometer (IRMS)

Isotope-ratio mass spectrometry (IRMS) is a type of mass spectrometry. The method uses an isotope-ratio mass spectrometer (IRMS) measure the relative abundance of isotopes in a given sample. For the methods and systems of the present invention, isotope-ratio mass spectrometry is used to measure or analyze the isotopic variations of stable isotopes in samples of interest. The isotope-ratio mass spectrometer (IRMS) allows the precise measurement of mixtures of naturally occurring isotopes. See, Townsend, A. (ed) (1995) Encyclopaedia of Analytical Science Encyclopaedia of Analytical Science. London: Academic Press Limited, which is incorporated by reference herein in its entirety.

Isotope-ratio mass spectrometers useful herein can be of either the magnetic sector design or the quadrupole design, with the magnetic sector design generally being preferable. The magnetic sector type, also known as the “Nier type”, after its designer Aired Nier, operates by ionizing the sample and accelerating it over a potential (usually in the kilo-volt range). The resulting stream of ions is thus separated according to their mass-to-charge ration, or m/z.

See, Goetz, A.; Platzner, I. T. (Itzhak Thomas); Habfast, K.; Walder, A. J. (1997). Modern isotope ratio mass spectrometry. London: J. Wiley, which is incorporated by reference herein in its entirety.

An example of an isotope-ratio mass spectrometer useful herein is a ThermoScientific DELTA V™ Plus Isotope Ratio Mass Spectrometer. See, http//www.thermoscientific.com/en/product/delta-v-plus-isotope-ratio-mass-spectrometer.html, which is incorporated by reference herein in its entirety.

Nuclear Magnetic Resonance (NMR) Spectrometer

A nuclear resonance (NMR) spectrometer is a very common analytical device that is even now available in many undergraduate chemistry laboratories. NMR spectroscopy is an analytical method that uses the magnetic properties of certain atomic nuclei to provide both qualitative and quantitative physical and chemical properties of atoms and the molecules in which they are contained. When placed in a magnetic field, various nuclei or isotopes, e.g., ¹H and ¹³C, absorb electromagnetic radiation at a frequency characteristic of the isotope. Such information can include structures, dynamics, chemical environment, and also isotope and isotope ratio information.

See V. Govindaraju, K. Young, and A. A. Maudsley, Proton NMR chemical shifts and coupling constant for brain metabolites. NMR in Biomedicine, Volume 13, Issue, pages 129-153, May 2000; and J. H. H. Nelson and J. H. Nelson, Nucelar Magnetic Resonance Spectroscopy: 1^{st} Edition, ISBN-13: 9780130334510, 2002, Prentice Hall, which are incorporated by reference herein in their entirety.

An example of a nuclear magnetic resonance spectrometer useful herein is a Thermo Scientific picoSpin 80 NMR Spectrometer.

Computerized Data System (CDS)

The systems of the present invention comprise a computerized data system (CDS). A computerized data system is the computer or computer system for collecting, processing, and storing the isotope ratio data generated from the sampling and collection of samples from the processes of the present invention. In many cases, the computerized data system is integrated into or closer associated with the isotope analyzer. In other others it is a separate or stand-alone computer, whether a hand-held, lap-top, desk-top, or main-frame computer which is attached or associated with the isotope analyzer. By associated is meant that the data from the isotope analyzer is either sent electronically, wirelessly, or transmitted via a separate storage device such as a CD or flash-drive.

Reactor

The processes described herein, whether chemical or biological, are generally conducted in some type of reactor or vessel. Although not strictly a component of the systems of the present invention, the reactor can in some embodiments be considered a component. In such cases, the systems of the present invention further comprise a reactor.

Chemical and biological reactors come in a wide array of forms varying from small size laboratory glassware such as test tubes and flasks, to scale-up and pilot plant systems, to large scale manufacturing plants. The reactor can be one used to conduct a discrete or single batch process or reaction. Alternatively, the reactor can be one that operates on a continuous basis wherein a feedstock of starting materials or reactants are continuously supplied and a reaction effluent or product stream is continuously removed. Such continuous reactors can operate on a flow or stream basis.

See R. Turton, R. C. Bailie, W. B. Whiting, J. A. Shaeiwita, and D. Bhattacharyya, Analysis, Synthesis and Design of Chemical Proceeses (4^{th} Edition) (Prentice Hall International Series in the Physical and Chemical Engineering Sciences), Jul. 2, 2012, which is incorporated by reference herein in its entirety.

### EXAMPLES

The following examples further describe and demonstrate embodiments within the scope of the present invention. The Examples are given solely for purpose of illustration and are not to be construed as limitations of the present invention, as many variations thereof are possible without departing from the spirit and scope of the invention.

### Example 1

### Method for Molecularly Isotopically Engineering Sitagliptin

Sitagliptin is an oral antihyperglycemic of the dipeptidyl peptidase-4 (DPP-4) inhibitor class. It is marketed as the phosphate salt under the trade name Januvia® as an antidiabetic drug. See, “Synthesis of Sitagliptin, the Active Ingredient in Januvia® and Janumet® ”, Jaume Balsells, Yi Hsiao, Karl B. Hansen, Feng Xu, Norihiro Ikemoto, Andrew Clausen, and Joseph D. Armstrong III, Chapter 5, pages 102-126 in Green Chemistry in the Pharmaceutical Industry, Edited by Peter J. Dunn, Andrew S. Wells and Michael T. Williams, 2010 WILEY-VCH Verlag GmbH & Co. KGaA, Weinheim, ISBN: 978-3-527-32418-7, which is incorporated by reference herein in its entirety.

Sitagliptin corresponds to the following chemical structure, Compound 5. The most commonly used form of sitagliptin is the phosphoric acid salt monohydrate, CAS Number 654671-77-9, Compound 6.

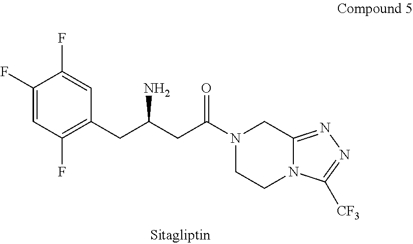 [SMILES: N[C@@H](CC(=O)N1CCn2c(nnc2C(F)(F)F)C1)Cc1cc(F)c(F)cc1F]

The multi-step chemical process shown below is used to prepare sitagliptin having a statistically defined isotopic composition. This example relies on the mass balance for the isotopes, although further isotopic fractionation effects for the reaction steps can be utilized. The synthesis is used to prepare sitagliptin target compounds having a δ¹³C statistical enrichment, of approximately −5‰ to +10‰ relative to the starting trifluoro carboxylic acid, Compound 1.

In step 1, the trifluoro carboxylic acid, Compound 1, is reacted to form the Meldrum acid adduct, Compound 2. Compound 2 is carried on without isolation in step 2 to the ketoamide, Compound 3. Compound 3 is further carried on without isolation in step 3 to the enamine, Compound 4. Compound 4 is asymmetrically hydrogenated in step 4 using a rhodium catalyst and the catalyst is removed to provide the sitagliptin free base, Compound 5. Compound 5 is converted to the phosphoric acid salt monohydrate, Compound 6, in step 5.

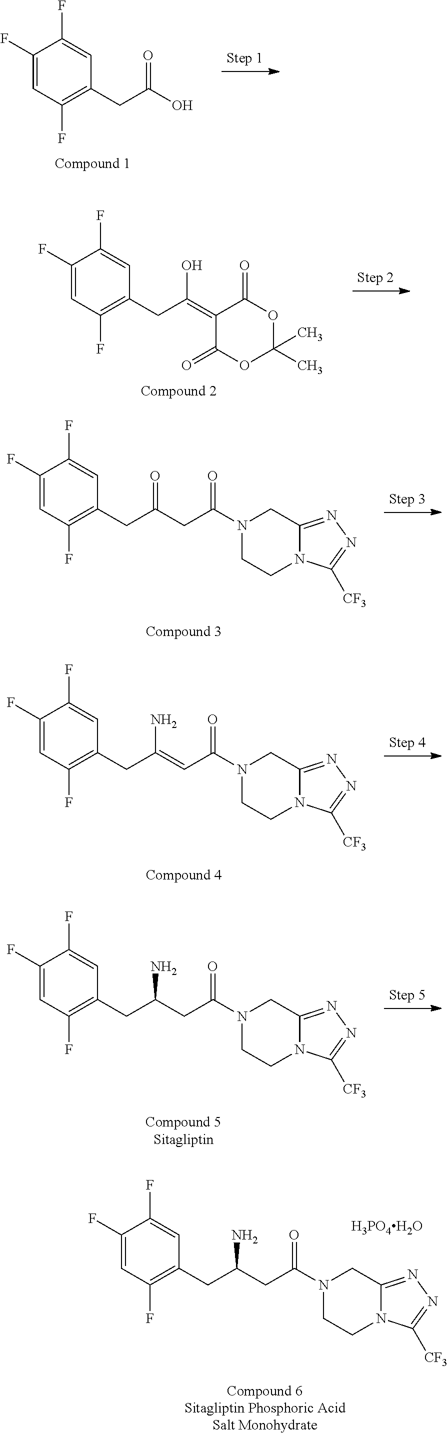

The following reaction conditions are used in the steps of the reaction scheme. Step 1: Pivaloyl cholide, Meldrum's acid (2,2-dimethyl-1,3-dioxane-4,6-dione, Compound 7), Hunigs base (N,N-diisopropylethylamine or DIPEA), and a catalytic amount, 8 mol %, of 4-dimethylaiminopyridine (DMAP), in acetonitrile.

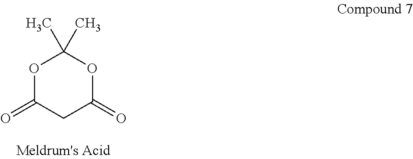

Step 2: Trifluoromethyl triazole (Compound 8), and a catalytic amount of trifluoroacetic acid (TFA).

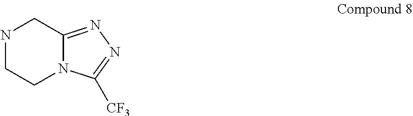 [SMILES: FC(F)(F)c1nnc2n1CCNC2]

Step 3: Methanol solution of ammonium acetate. Step 4: Asymetric hydrogenation using the in situ generated complex from 0.15 mol % of cyclooctadiene rhodium chloride dimer [Rh(COD)Cl₂]₂ and 0.31 mol % of t-butyl Josiphos ligand, with 100 psig H₂ in methanol at 50° C., followed by removal of the dissolved rhodium by Ecosorb C-941, an activated carbon/polyethylene homopolymer material available from Graver Technologies. The sitagliptin is recrystallized from isopropyl alcohol/heptane. Step 5: Treatment with phosphoric acid in water and isopropyl alcohol to provide the phosphoric acid salt monohydrate.

Starting with the ¹³C isotopic compositions for the trifluoro carboxylic acid starting compound (Compound 1), Meldrum's acid (Compound 7), and the trifluoromethyl triazole (Compound 8), as indicated in Table 2, the ¹³C statistically defined isotopic compositions of sitagliptin (Compound 5) are obtained, and also reported as the δ¹³C statistical enrichment relative to Compound 1. Note that the isotopic composition is directly proportional to the number of atoms in a given compound, as expressed by the laws of mass balance (equation 2) and isotopic mass balance (equation 3). In this case for ¹³C, the starting trifluoro carboxylic acid (Compound 1) has 8 carbon atoms, whereas the target sitagliptin (Compound 5) has 16 carbon atoms. Also, Meldrum's acid (Compound 7) contributes 2 carbon atoms and the trifluoromethyl triazole (Compound 8) contributes 6 carbon atoms to the sitagliptin molecule. Note that similar mass balance analyses can be made for other elements such as hydrogen, oxygen, etc. and the reactants from where they are derived. The mass balance analysis can also be made to consider two or more different elements simultaneously. Table 2 provides a mass balance analysis for various ¹³C isotopic compositions for sitagliptin (Compound 5), considering various isotopic compositions for the starting material, namely Compound 1, and the reactants, Compound 7 and Compound 8, and the δ¹³C Statistical Enrichment compared to Compound 1.

TABLE 2
¹³C Isotopic Compositions

| | Starting | | | | |
|---|---|---|---|---|---|
| | Material | Reactant | Reactant | Sitagliptin | δ¹³C |
| Exam- | (Com- | (Com- | (Com- | (Com- | Statistical |
| ple No. | pound 1) | pound 7) | pound 8) | pound 5) | Enrichment¹ |
| 1 | −10.00‰ | 0.00 | 0.00 | −5.00‰ | +5.00‰ |
| 2 | −20.00‰ | 0.00 | 0.00 | −10.00‰ | +10.00‰ |
| 3 | −10.00‰ | −20.00‰ | −15.00‰ | −13.13‰ | −3.13‰ |
| 4 | −10.00‰ | −15.00‰ | −20.00‰ | −14.38‰ | −4.38‰ |
| 5 | −20.00‰ | −10.00‰ | −15.00‰ | −16.88‰ | +3.12‰ |
| 6 | −30.00‰ | −12.00‰ | −20.00‰ | −24.00‰ | +6.00‰ |

¹δ¹³C statistical enrichment (a positive value indicates enrichment, a negative value indicates depletion, and zero indicates no difference) compared to starting material (Compound 1)

The method and system described herein are useful for preparing sitagliptin having a target statistically defined isotopic composition. The foregoing example illustrates the applicable mass balance and isotopic mass balance laws. Other ¹³C statistically defined enriched sitagliptin compositions can be achieved by selecting other ¹³C compositions for the starting Compound 1 and the reactants (Compound 7 and Compound 8) or by using a different starting compound and reactants and/or a different overall reaction scheme. Also, sitapliptin having statistically defined enrichment for other stable isotopes and combinations of isotopes can be prepared.

The methods and systems of the present invention are useful for preparing statistically defined isotopic compositions for a wide variety of other chemical or biological substances.

### Example 2

### Method and System for Continuous Monitoring of Reaction Yield via Online Stable-Isotope Ratio Monitoring Using a Cavity Ring-Down Spectrometer (CRDS)

Natural-abundance stable-isotope ratios are quantitatively related to the yield of reactions. See, J. P. Jasper, L. E. Wearier, and J. M. Hayes, Process Patent Protection: Characterizing Synthetic Pathways by Stable-Isotope Measurements, Pharmaceutical Technology, 2007, 31(3):68-73, which is incorporated by reference herein in its entirety. Until recently, the monitoring of such ratios was typically preformed offline (e.g., J. P. Jasper, T. M. Schelhorn, and J. L. Treadway, A Stable Isotope Indirect calorimeter for the Quantification of the Metabolic Rate of ¹³C-Labelled Metabolites in Mice, Abstract from The International Isotope Society, King of Prussia, Pa.: Oct. 27, 2000, which is incorporated by reference herein in its entirety. With the recent advent of continuous isotope-ratio monitoring via Cavity Ring-Down Spectroscopy we employ a Picarro CRDS G2131-i Analzer to monitor the isotopic composition (e.g., δ¹³C) as a quantitative index of yield.

For this continuous isotope ratio mass spectrometry experiment, real-time monitoring of reaction yield in production is a key parameter. For initial, independent calibration of yield, the integrated output of an online flow meter and an online pCO₂ (i.e. a partial press CO₂) meter (LI-800 CO2 Gas Hound, LI-COR Inc., Lincoln, Nebr., U.S.A.) permits a real-time estimate of the mass of CO₂ generated by the reactor up to the point of total yield. The present development of continuous, online isotope-ratio mass instruments presents an opportunity to monitor reaction yield in real time via the isotopic composition of the off gas from the reactor. We perform an experiment to illustrate the utility of natural-abundance stable isotopes in process chemistry. We use a reaction system that generates a product such as carbon dioxide, such as from a beer brewing system. Alternatively, we use a reaction system that generates carbon dioxide from a pharmaceutical manufacturing process, e.g., the removal of a BOC protecting group from a pharmaceutical product intermediate which has been protected with a BOC protecting group via di-tert-butyl dicarbonate (note that the BOC protecting group is generally used to protect amino groups). See, S. Doherty and A. Garrett, Another Tool from the PAT Tool Box, Spectrometry: Issue 4, Nov. 11, 2005, European Pharmaceutical Review, which is incorporated by reference herein in its entirety.

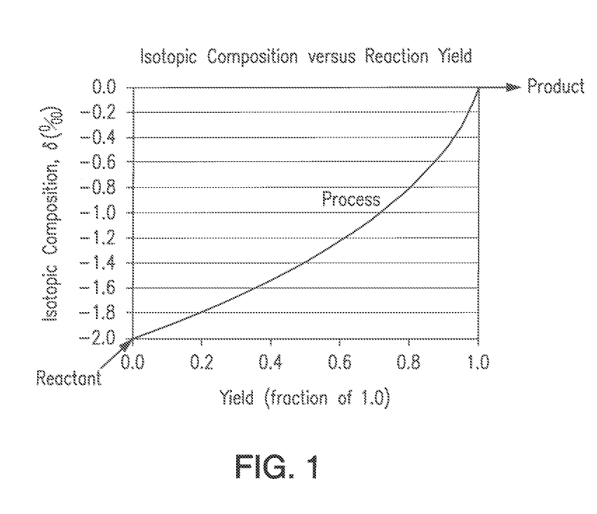

We employ a Picarro CRDS G2131-i Analyzer and a laptop computer to continuously monitor the δ¹³C—CO₂ generated in these reactions. FIG. 1 depicts the isotopic composition of such a reaction product plotted as a function of reaction yield. The isotopic composition, δ, increases as the reaction yield approaches 1, that is, as it approaches completion.

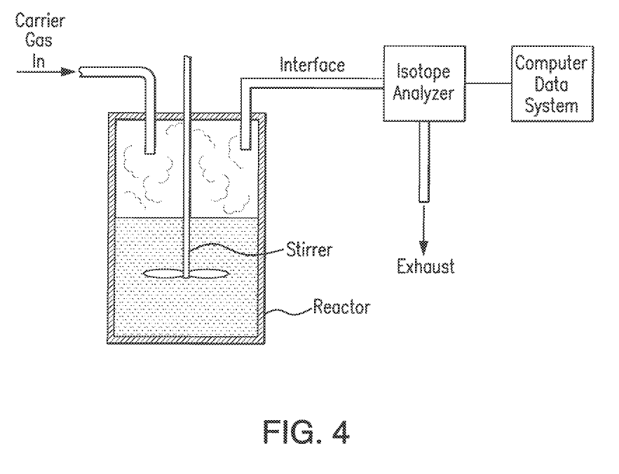

FIG. 4 depicts a system for continuously monitoring the progress of such a chemical process as per this Example 2, in which a gaseous product (or by-product, e.g., CO₂) is generated, e.g., the production of CO₂ from a fermentation process or a BOC deprotection reaction. This system illustrates a stirred reactor, a line for blowing a carrier gas (e.g., helium, nitrogen, or the like) through the system to continuous sample it to collect the gaseous product or by-product (e.g., CO₂), and an effluent tube which feeds in to an isotope analyzer and an associated computerized data system (CDS). The interface is essentially the connection of the effluent tube to the mass spectrometer. The progress of the fermentation is monitored via isotope information from the ¹³C/¹²C ratio of the CO₂ produced. Alternatively, the progress of the fermentation is monitored from the ¹⁸O/¹⁶O ratio of the CO₂ produced.

Alternatively, the method and system of Example 1 is used to monitor the progress of a reaction in the synthesis of a pharmaceutical product. In this case the progress of a BOC deprotection reaction of a pharmaceutical intermediate is monitored. The pharmaceutical intermediate is prepared via reaction of the desired precursor with a standard BOC reagent such as di-tert-butyl dicarbonate.

The progress of the deprotection reaction is monitored via the carbon dioxide that is liberated during the deprotection reaction by determining the ¹³C/¹²C ratio or the ¹⁸O/¹⁶O ratio of the CO₂ produced during the deprotection reaction.

The method and system described herein are useful for continuously monitoring the progress or reaction yield of a chemical or a biological process.

### Example 3

### Method and System for Continuous Monitoring of Reaction Yield via Online Stable-Isotope Ratio Monitoring Using an Isotope Ratio Mass Spectrometer (IRMS)

The method and system of Example 3 is essentially the same as for Example 2, except that an Isotope Ratio Mass Spectrometer (IRMS), such as a ThermoScientific DELTA V™ Plus Isotope Ratio Mass Spectrometer, is employed in place of the cavity ring-down spectrometer (CRDS).

The method and system described herein are useful for continuously monitoring the progress or reaction yield of a chemical or a biological process.

### Example 4

### Method and System for Continuous Monitoring of Reaction Yield via Online Stable-Isotope Ratio Monitoring Using a Nuclear Magnetic Resonance (NMR) Spectrometer

The method and system of Example 4 is essentially the same as for Example 2, except that a Nuclear Magnetic Resonance (NMR) Spectrometer, such as a Thermo Scientific picoSpin 80 NMR Spectrometer is employed in place of the cavity ring-down spectrometer (CRDS).

The method and system described herein are useful for continuously monitoring the progress or reaction yield of a chemical or a biological process.

### Example 5

### Method for Molecularly Isotopically Engineering Naproxen

Naproxen is a nonsteroidal anti-inflammatory drug (NSAID) of the proprionic acid class. Naproxen is commonly used for relief of a wide variety of pain, swelling, inflammation, and fever.

Naproxen, racemic or (±), corresponds to the following chemical structure, Compound 15, and has the chemical name 2-(6-methoxynaphthalene-2-yl)propanoic acid.

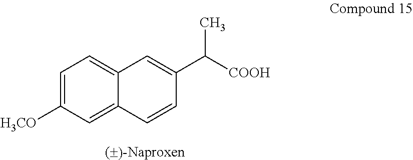 [SMILES: COc1ccc2cc(C(C)C(=O)O)ccc2c1]

Naproxen has a chiral center and can exist as a mixture of enantiomers. The (S) enantiomer which has a (+) optical rotation, namely (+)-(S)-naproxen (Compound 16), is the active drug form, which is usually sold as the sodium salt. (+)-(S) Naproxen corresponds to CAS Number 22204-53-1.

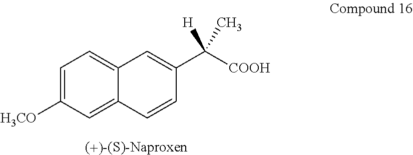 [SMILES: COc1ccc2cc([C@H](C)C(=O)O)ccc2c1]

The multi-step chemical process shown below is used to prepare naproxen having a statistically defined isotopic composition. This multi-step process is the industrial process reported by Syntex. See, Harrington P J, Lodewijk E (1997). “Twenty Years of Naproxen Technology”. Org. Process Res. Dev. 1 (1): 72-76 (1997), which is incorporated by reference herein in its entirety. This example relies on the mass balance for the isotopes, although further isotopic fractionation effects for the reaction steps can be utilized. The synthesis is used to prepare naproxen target compounds having a δ¹³C statistical enrichment, of approximately −5‰ to +10‰ relative to 2-bromo-6-methoxynaphthalene, Compound 13, as the reference starting material.

In step 1, 2-naphthol, Compound 9, is brominated form the dibromo naphthol, Compound 10. In step 2 Compound 10 is treated with sodium bisulfate, NaHSO₃, to form 2-bromo-6-hydroxynaphthalene, Compound 11. Compound 11 is converted in step 3 to the ether, 2-bromo-6-methoxynaphthalene, Compound 13, by reacting with methyl chloride (Compound 12) in the presence of a base. In step 4 Compound 13 is first treated with magnesium and reacted with the 2-bromopropionic acid based compound, Compound 14, via a Grignard reaction to form racemic (±)-naproxen, Compound 15. In step 5, the racemic (±)-naproxen is resolved by the Pope-Peacheay method to provide the desired (+)-(S)-naproxen, Compound 16. See, Pope, W. J.; Peachey, S. J. The application of powerful optically active acids to the resolution of externally compensated basic substances. Resolution of tetrahydroquinaldine. J. Chem. Soc. Trans. 1899, 75, 1066-1093, which is incorporated by reference herein in its entirety.

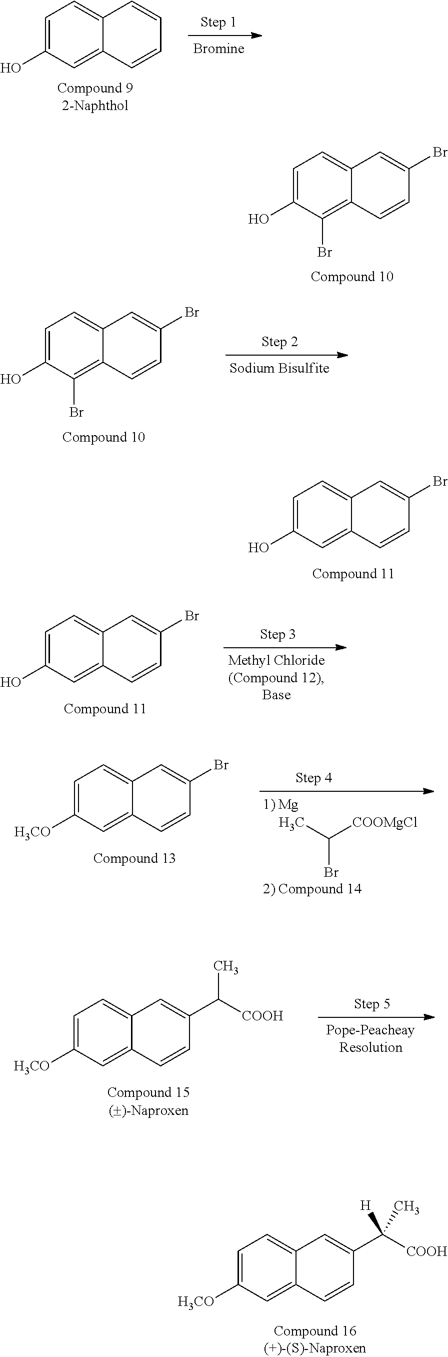

Pre-selection of the stable-isotopic compositions of the starting material 2-bromo-6-methoxynaphthalene (Compound 13), e.g. δ¹³C⁻−12‰, −22‰, −32‰ vs. VPDB (Vienna Peedee Belemnite) yields the product of discrete stable-isotopic ranges (X±x‰, Y±y‰, Z±z‰ vs. VPDB).

It should be noted that the synthetic scheme, above, begins with 2-naphthol (Compound 9). However, for this example the relative isotopic compositions are determined with respect to the 2-bromo-6-methoxynaphthalene (Compound 13) and focus on step 4 and step 5 of the synthetic scheme. It should be recognized that other compounds in the synthetic scheme can be used as a point of reference for a starting point.

Starting with the ¹³C isotopic compositions for the starting compound 2-bromo-6-methoxynaphthalene (Compound 13), and the 2-bromopropionic acid Grignard reagent, (Compound 14), as indicated in Table 3, the ¹³C statistically defined isotopic compositions of (+)-(S)-naproxen (Compound 16) are obtained, and also reported as the δ¹³C statistical enrichment relative to Compound 13. Note that the isotopic composition is directly proportional to the number of atoms in a given compound, as expressed by the laws of mass balance (equation 2) and isotopic mass balance (equation 3). In this case for ¹³C, the starting 2-bromo-6-methoxynaphthalene (Compound 13) has 11 carbon atoms, whereas the target (+)-(S)-naproxen (Compound 16) has 14 carbon atoms. Also, the 2-bromopropionic acid Grignard reagent (Compound 14) contributes 3 carbon atoms to the (+)-(S)-naproxen molecule. Note that similar mass balance analyses can be made for other elements such as hydrogen, oxygen, etc. and the reactants from where they are derived. The mass balance analysis can also be made to consider two or more different elements simultaneously. Table 3 provides a mass balance analysis for various ¹³C isotopic compositions for (+)-(S)-naproxen (Compound 16), considering various isotopic compositions for the reference starting material, namely Compound 13, and the reactant, Compound 14, and the δ¹³C Statistical Enrichment compared to Compound 13.

TABLE 3
¹³C Isotopic Compositions

| | Starting | | (+)-(S)- | δ¹³C |
|---|---|---|---|---|
| Exam- | Material | Reactant | Naproxen | Statistical |
| ple No. | (Compound 13) | (Compound 14) | (Compound 16) | Enrichment¹ |
| 1 | −10.00‰ | 0.00 | −7.86‰ | +2.14‰ |
| 2 | −20.00‰ | 0.00 | −15.71‰ | +4.29‰ |
| 3 | −10.00‰ | −20.00‰ | −12.14‰ | −2.14‰ |
| 4 | −10.00‰ | −15.00‰ | −11.07‰ | −1.07‰ |
| 5 | −20.00‰ | −10.00‰ | −17.86‰ | +2.14‰ |
| 6 | −30.00‰ | −12.00‰ | −26.14‰ | +3.86‰ |

¹δ¹³C statistical enrichment (a positive value indicates enrichment, a negative value indicates depletion, and zero indicates no difference) compared to starting material (Compound 13).
Values are typically vs. VPDB (Vienna Peedee Belemnite).

The method and system described herein are useful for preparing (+)-(S)-naproxen (Compound 16) having a target statistically defined isotopic composition. The foregoing example illustrates the applicable mass balance and isotopic mass balance laws. Other ¹³C statistically defined enriched naproxen compositions can be achieved by selecting other ¹³C compositions for the starting Compound 13 and the reactant (Compound 14) or by using different starting compound and reactant(s) and/or a different overall reaction scheme. Also, (+)-(S)-naproxen (Compound 16) having statistically defined enrichment for other stable isotopes and combinations of isotopes can be prepared.

The methods and systems of the present invention are useful for preparing statistically defined isotopic compositions for a wide variety of other chemical or biological substances.

### Example 6

### Method for Molecularly Isotopically Engineering Naproxen

The following is a further example for molecularly isotopically engineering naproxen. This example illustrates the manufacture of naproxen of predetermined, i.e. designed, stable isotopic compositions. These compositions can be useful for identity and security protection. This example is focused on the last two steps (steps 4 and 5) of the naproxen synthesis as described in Example 5, above, and specifically provides data for the synthesis of (±) naproxen from the first of those last two steps. The ether compound, 2-bromo-6-methoxynaphthalene, compound 13, is first treated with magnesium and reacted with the 2-bromopropionic acid based compound, Compound 14, via a Grignard type reaction to form racemic (±)-naproxen, Compound 15. The racemic (±)-naproxen can be further resolved to provide the desired (+)-(S)-naproxen, Compound 16 (e.g. with an N-alkylglucamine), or alternatively via an enzymatic resolution.

Molecular Isotopic Engineering (MIE) is the directed stable-isotopic synthesis of chemical products, e.g. for reasons of product identification and of product security. We report here a correspondence between the observed and predicted stable-isotopic results [δ¹³C, δ¹⁸O, and δD (δ²H)] for a directed synthesis of a racemic mixture from its immediate precursors. The observed carbon-isotopic results are explained by the laws of mass balance and isotope mass balance. By contrast, the oxygen and hydrogen isotopic results require an additional assessment of the effects of O and H exchange, presumably due to interaction with water in the reaction solution.

A previous, cooperative study with the US FDA, Division of Pharmaceutical Analysis (DPA) showed that individual manufacturers of naproxen could readily be differentiated by their stable-isotopic provenance, from δ¹³C, δ¹⁸O, and δD. See, Wokovich, A. M., J. A Spencer, B. J. Westenberger, L. F. Buhse, and J. P. Jasper. (2005) Stable isotopic composition of the active pharmaceutical ingredient (API) Naproxen. J. Pharm. Biomed. Anal., 38:78.1-784. Results from two out of three of the naproxen samples produced for this study correspond to the naproxen results observed in the cooperative study. A third does not as readily correspond to any of the samples observed in the cooperative study because no associated naproxen appears to have been produced from the requisite starting material. The general correspondence of the present and previous naproxen data illustrates the fidelity of molecular isotopic engineering (MIE). We suggest that ME can be readily employed in the bio/pharmaceutical industry without alteration of present manufacturing processes other than isotopically designing and selecting and/or monitoring reactants and products.

Product identity, product security, and intellectual property protection remain major concerns in the bio/pharmaceutical industry (Jasper, 2004; Saha and Bhattacharya, 2011; Basta, 2015). The directed stable-isotopic synthesis of chemical products allows the predetermination of the stable-isotopic composition of materials to address these challenges (Jasper et al., 2015a). Product identification is typically performed at three levels: overt, covert, and forensic levels (e.g., Jasper, 2004). We focus here on a forensic or analytical approach: the analysis of natural-abundance stable isotopes in these products. Natural-abundance stable isotopes are natural tracers that occur in all matter (e.g., Hoefs, 1997).

Early work in cooperation with the US FDA on the product characterization of naproxen revealed manufacturer-level isotopic provenance of this small analgesic molecule (Wokovich et al., 2005), which was referred to as “The Manufacturer's Fingerprint.” This isotopic provenance represented the convergence of the effects of the stable-isotopic compositions of starting materials and isotopic effects of the synthetic process. Rather than merely accepting the random effects of variable sourcing and synthetic process on the stable-isotopic compositions of products, here we take a proactive approach to purposefully direct the stable-isotopic composition of bio/pharmaceutical products. The main rationale for MIE is to pro-actively design the isotopic ranges of products for reasons of product identification and of product security. As an example of MIE, we analyzed the isotopic products of a later step of a naproxen synthesis:

2-Bromo-6-Methoxynapthalene+Bromopropionate→(±)Naproxen

Pre-selection of three different stable-isotopic compositions of the starting material, 2-bromo-6-methoxynaphthalene, respectively yielded racemic naproxen products of three discrete stable-isotopic ranges. The resulting MIE naproxen is very different from a naproxen molecule that has merely been substituted at a single position with a different isotope as in deuterium labeling (Tung, 2010 and refs. therein). Our directed isotopic synthesis is just one example of MIE to predetermine the discrete isotopic ranges of bio/pharmaceutical products. In principle, the MIE approach should be readily adapted to existing bio/pharmaceutical manufacturing units. The adjustment to an existing manufacturing process would be the use of starting materials or synthetic intermediates of pre-selected or pre-measured stable-isotopic compositions. The manufacturing apparatus would remain unchanged. This approach could have broad application in securing drug identity/provenance from manufacturing plant to consumer.

By way of background, we have developed four generations of stable-isotopic methods and technologies: (i) product characterization (for both small molecules and biologics) [e.g., Jasper, 2007; Jasper et al., 2004, 2015a; Wokovich et al., 2005], (ii) process characterization (notably, for process protection or integrity), [Jasper et al., 2007: Martin et al. 2008], (iii) in-process (continuous) analysis [WO 2015/103183 A1, to Jasper, published Jul. 9, 2015], and now (iv) molecular isotopic engineering or MIE

Early work in cooperation with the US FDA on the product characterization of naproxen revealed manufacturer-level isotopic provenance of this small analgesic molecule (Wokovich et al., 2005) which was referred to as “The Manufacturer's Fingerprint.” This isotopic provenance represented the convergence of the effects of the stable-isotopic compositions of starting materials and isotopic effects of the synthetic process. Rather than merely accepting the random effects of variable sourcing and synthetic process on the stable-isotopic compositions of products, we take a proactive approach to purposefully design and predetermine the stable-isotopic composition of bio/pharmaceutical products. The main rationale for MIE is to design the isotopic ranges of products for reasons of product identification and of product security.

Samples: Three groups of samples were analyzed here to examine the natural-abundance stable-isotopic compositions for naproxen synthesis, including the two reactants (2-bromo-6-methoxynaphthalene and bromopropionic acid) and the end product (racemic naproxen).

Naproxen Synthesis: A late-stage synthesis of naproxen was performed according to the following Grignard Reagent reaction starting with compound 13, 2-bromo-6-methoxynaphthalene.

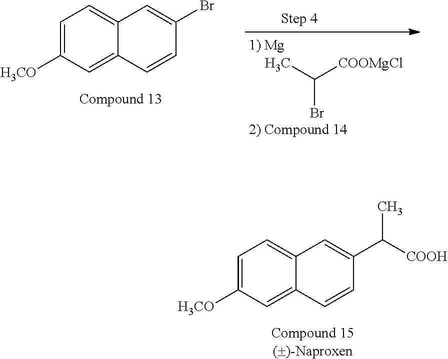

Reactants. Eight samples of 2-bromo-6-methoxynaphalene were obtained from a worldwide selection of suppliers (Table 4). A Grignard reagent (bromopropionic acid) was acquired from Sigma-Aldrich (St. Louis, Mo. USA). Three of the 2-bromo-6-methoxynaphalene samples, from Sample Source 1 (Alfa Aesar), Sample Source 2 (Combi-Blocks) and Sample Source 3 (Matrix), were obtained and analyzed and specifically selected for this study based on their differing ¹³C compositions: one high, one low, and one intermediate ¹³C composition. Samples of the product (racemic naproxen) were synthesized from the three different starting materials.

Grignard Formation: 2-bronco-6-methoxynaphthalene was dissolved in a round bottom flask of anhydrous toluene and anhydrous tetrahydrofuran (THF) with heating and degassing. The 2-bromo-6-methoxynaphthalene solution was added dropwise to the magnesium via addition funnel. The resultant Grignard solution was allowed to cool to room temperature under nitrogen.

Magnesium Salt Formation on Bromopropionic Acid: Alpha-bromopropionic, acid was dissolved in anhydrous THF. The solution was cooled in a dry ice/acetone bath to −15° C. and methyl magnesium chloride was added via syringe while maintaining the temperature below 0° C. The temperature was kept below 0° C. until the solution was used.

Coupling Reaction: The Grignard solution was transferred into a two-neck round bottom flask with a thermometer and a septum via cannula, then was degassed. The solution was cooled in an ice bath and the mixed magnesium halide salt complex was added via cannula maintaining the temperature at 15-20° C. The reaction was stopped after 2 hr and was cooled in an ice bath and a solution of 10 mL of 12N HCl in 75 mL of water was added. After stirring for 5 min, the biphasic mixture was filtered, and the filter was washed with 25 mL of toluene and 25 mL of water. The layers were separated and the organic phase was extracted with 2×75 mL of 10% NaOH solution. The basic extracts were combined, washed with toluene (˜25 mL) and filtered. To the filtrate was added 7.5 mL, of methanol and 6 mL of toluene. This mixture was then acidified with concentrated HCl to pH 5. The resulting slurry was heated to reflux for 1 hr and allowed to cool overnight with stirring. The product was washed with 10 mL of water, 2×2 mL of toluene and 2×2 mL of hexane and then dried to give an off-white solid. After drying under high vacuum for 48 hr there was 5.8828 g (53% yield).

Stable-Isotopic Analyses: Three stable-isotopic measurements (δ¹³C, δ¹⁸O and δD) were made of each of the components of this study. In the starting-material survey study, three stable-isotope ratios were measured on each of the eight samples of 2-bromo-6-methoxynapthalene triplicate analysis (i.e., 8 batches×3 isotope ratios×3 replication=72 measurements) to assess analytical precision (Jasper, 2001; Jasper et al., 2005). Nine analogous isotopic measurements were made on the bromopropionic acid reagent (3 isotope ratios×3 replications). Triplicate analyses were also performed for each of the three isotope ratios of the five batches of naproxen synthesized, yielding 45 isotope measurements. Thus, a total of 126 stable-isotopic measurements of the samples were performed in this study.

Carbon and Oxygen Isotope Analyses: As detailed elsewhere (Jasper et al., 2015), carbon (δ¹³C) and oxygen (δ¹⁸O) isotopic analyses were performed respectively on (i) a Carlo Erba 1108 Elemental Analyzer interfaced using a Conflo III interface to a Thermo Scientific Delta V isotope ratio mass spectrometer (EA/IRMS) and (ii) a Finnigan Thermal Conversion/Elemental Analyzer (TCEA) interfaced to Finnigan Delta V Plus isotope-ratio mass spectrometer (thus a TCEA/IRMS).

Hydrogen (δD) Isotopic Analyses: Hydrogen that is not bound to carbon in a molecule may readily exchange with other hydrogen atoms present in ambient moisture (i.e., H₂O). This exchange happens even at room temperature and is difficult to control. To generate precise δD values for a given compound, the exchangeable hydrogen portions must be accounted for or controlled. Samples are weighed into individual 3.5 mm×5 mm silver “boats” and equilibrated with reference waters of known δD values to calculate the amount of exchangeable hydrogen in the sample (Meier-Augenstein et al., 2011). The samples are allowed to equilibrate for two hours at 50° C. inside a container with an aliquot of calibrated reference water. The equilibration process is repeated twice on separate aliquots of sample using reference water samples that have a difference of −233‰ in δD value. After equilibration the samples are dried overnight in a vacuum oven at 50° C., then immediately transferred to a Costech Zero Blank autosampler of a Finnigan MAT, Thermal Conversion Elemental Analyzer (TC/EA) and evacuated to remove ambient moisture. Several reference standards accompany each batch of samples, including a polyethylene standard that has no exchangeable hydrogen and is therefore unaffected by ambient moisture. In the TC/EA the samples are reduced at 1400° C. in the presence of glassy carbon. The resulting hydrogen is then separated from other gases via a gas chromatograph and advected into an isotope ratio mass spectrometer (IRMS) for isotopic analysis to obtain the δD values. Using post-analysis calculations (Meier-Augenstein et al., 2011), the δD value of the non-exchangeable hydrogen can be quantified from the equilibrated sample data sets.

Units of Stable Isotopic Measurement: Carbon (and all other) isotopic results are expressed in δ values (‰=parts per thousand differences from international standards) defined as:

δ(‰)=([(Rsmpl)/(Rstd)]−1)×(1000)

where Rsmpl=the ¹³C/¹²C ratio of the sample material and R_{std}=the ¹³C/¹²C ratio of an International Atomic Energy Authority standard (IAEA, known as Vienna Peedee Belemnite (VPDB) whose ¹³C/¹²C ratio has been defined as the official zero point of the carbon-isotopic scale). ¹⁸O/¹⁶O and D/H values are given relative to IAEA Vienna Standard Mean Ocean Water (VSMOW) standard which gives the zero points of the oxygen and hydrogen-isotopic scales.

Estimates of Uncertainty: Since all measurements in this study were made in triplicate, the averages and 1σ-standard deviations are reported here for the observed isotopic data in Tables 4 and 5. Two sigma standard deviations are shown for the deviations from mass balance and isotope mass balance.

Characteristic one sigma (1σ) standard deviations for the isotope measurements reported in this study were: δ¹³C (±0.03‰), δ¹⁸O (±0.09‰), and δD (±1.0‰) as shown in Table 4.

Stable-Isotopic Composition of Reactants: The δ¹³C, δ¹⁸O, and δD compositions of eight samples of the reactant 2-bromo-6-methoxy-naphalene measured in triplicate are shown in Table 4.

TABLE 4
Stable Isotopic Compositions of 2-Bromo-6-Methoxynaphthalene

| | | | | δ¹³C | ±1σ | δ¹⁸O | ±1σ | δD | ±1σ |
|---|---|---|---|---|---|---|---|---|---|
| Sample | δ¹³C | δ¹⁸O | δD | ‰vs. | Std. Dev. | ‰vs. | Std. Dev. | ‰ vs. | Std. Dev. |
| Name/ | ‰vs. | ‰vs. | ‰vs. | VPDB | ‰ | VSMOW | ‰ | VSMOW | ‰ |
| Number | VPDB | VSMOW | VSMOW | Statistics | | | | | |
| Matrix | −23.97 | 24.24 | −116.6 | | | | | | |
| Scientific | | | | | | | | | |
| W12M/1 | | | | | | | | | |
| Matrix | −24.02 | 24.31 | −114.3 | −24.01 | 0.04 | 24.24 | 0.07 | −115.1 | 1.3 |
| Scientific | | | | | | | | | |
| W12M/2 | | | | | | | | | |
| Matrix | −24.04 | 24.17 | −114.3 | | | | | | |
| Scientific | | | | | | | | | |
| W12M/3 | | | | | | | | | |
| AK | −23.97 | 24.17 | −115.7 | | | | | | |
| Scientific | | | | | | | | | |
| LC33871/1 | | | | | | | | | |
| AK | −23.99 | 24.27 | −116.4 | −24.01 | 0.05 | 24.25 | 0.07 | −116.2 | 0.5 |
| Scientific | | | | | | | | | |
| LC33871/2 | | | | | | | | | |
| AK | −24.06 | 24.30 | −116.6 | | | | | | |
| Scientific | | | | | | | | | |
| LC33871/3 | | | | | | | | | |
| Oakwood | −24.48 | 13.77 | −64.2 | | | | | | |
| Chemical | | | | | | | | | |
| D13F/1 | | | | | | | | | |
| Oakwood | −24.53 | 13.76 | −68.0 | −24.49 | 0.04 | 13.77 | 0.02 | −65.9 | 1.9 |
| Chemical | | | | | | | | | |
| D13F/2 | | | | | | | | | |
| Oakwood | −24.46 | 13.80 | −65.6 | | | | | | |
| Chemical | | | | | | | | | |
| D13F/3 | | | | | | | | | |
| Sigma | −24.51 | 13.86 | −70.6 | | | | | | |
| Aldrich | | | | | | | | | |
| MKBR4254V/1 | | | | | | | | | |
| Sigma | −24.49 | 13.95 | −68.6 | −24.50 | 0.01 | 13.96 | 0.10 | −69.2 | 1.2 |
| Aldrich | | | | | | | | | |
| MKBR4254V/2 | | | | | | | | | |
| Sigma | −24.49 | 14.07 | −68.6 | | | | | | |
| Aldrich | | | | | | | | | |
| MKBR4254V/3 | | | | | | | | | |
| Combi | −28.76 | 0.36 | −65.4 | | | | | | |
| Blocks | | | | | | | | | |
| L74583/1 | | | | | | | | | |
| Combi | −28.70 | 0.54 | −65.0 | −28.73 | 0.03 | 0.45 | 0.09 | −64.5 | 1.0 |
| Blocks | | | | | | | | | |
| L74583/2 | | | | | | | | | |
| Combi | −28.75 | 0.45 | −63.5 | | | | | | |
| Blocks | | | | | | | | | |
| L74583/3 | | | | | | | | | |
| TCIC¹ Ltd. | −29.67 | −2.45 | −144.9 | | | | | | |
| GJ01- | | | | | | | | | |
| CSBE/1 | | | | | | | | | |
| TCIC¹ Ltd. | −29.65 | −2.38 | −146.4 | −29.67 | 0.03 | −2.42 | 0.04 | −145.9 | 0.8 |
| GJ01- | | | | | | | | | |
| CSBE/2 | | | | | | | | | |
| TCIC¹ Ltd. | −29.71 | −2.44 | −146.3 | | | | | | |
| GJ01- | | | | | | | | | |
| CSBE/3 | | | | | | | | | |
| Appollo | −29.82 | 3.84 | −116.5 | | | | | | |
| Scientific | | | | | | | | | |
| Ltd. AS44- | | | | | | | | | |
| 7149/1 | | | | | | | | | |
| Appollo | −29.85 | 3.89 | −116.5 | −29.85 | 0.02 | 3.80 | 0.11 | −116.7 | 0.4 |
| Scientific | | | | | | | | | |
| Ltd. AS44- | | | | | | | | | |
| 7149/2 | | | | | | | | | |
| Appollo | −29.87 | 3.68 | −117.1 | | | | | | |
| Scientific | | | | | | | | | |
| Ltd. AS44- | | | | | | | | | |
| 7149/3 | | | | | | | | | |
| Alfa Aesar | −29.90 | 13.79 | −109.0 | | | | | | |
| 10137505/1 | | | | | | | | | |
| Alfa Aesar | −29.84 | 14.13 | −111.0 | −29.88 | 0.04 | 14.07 | 0.25 | −110.2 | 1.1 |
| 10137505/2 | | | | | | | | | |
| Alfa Aesar | −29.91 | 14.28 | −110.5 | | | | | | |
| 10137505/3 | | | | | | | | | |

¹TCIC (Tokyo Chemical Industry Company)

The δ¹³C, δ¹⁸O, and δD compositions of three samples of the reactant 2-bromopropionic acid measured in triplicate are shown in Table 5.

TABLE 5
Stable Isotopic Composition of 2-Bromopropionic Acid

| Sample Name/ | δ¹³C | δ¹⁸O | δD |
|---|---|---|---|
| Number | ‰ vs. VPDB | ‰ vs. VSMOW | ‰ vs. VSMOW |
| 2-bromopropionic | −30.05 | 11.01 | −19.0 |
| acid/1 | | | |
| 2-bromopropionic | −30.13 | 10.84 | −20.8 |
| acid/2 | | | |
| 2-bromopropionic | −30.21 | 11.35 | −21.1 |
| acid/3 | | | |
| Average | −30.13 | 11.07 | −20.3 |
| Standard | 0.08 | 0.26 | 1.1 |
| Deviation | | | |

The Stable-Isotopic Records of Naproxen Synthesis: The directed stable-isotopic synthesis of naproxen is discussed in two parts: the mass-balance/isotope-mass balance (MB/IMB) component, and then the deviations (if any significant) from MB/IMB. Since these results are compared to the MB/IMB frame of reference, that topic is briefly described here.

Frame of Reference: Comparison of Observed versus Predicted Isotopic Values: The laws of mass balance and isotope mass balance (e.g., in Jasper et al., 2005) are a primary frame of reference for assessing the results of the naproxen isotopic synthesis. The basic mathematics of MB/IMB (detailed in Jasper et al., 2007 and references therein) are summarized here:

Mass Balance: n_{A}+n_{B}=n_{C}

Isotope Mass Balance: n_{A}δ_{A}+n_{B}δ_{B}=n_{C}δ_{C}

Isotopic Fractionation (one component in excess):

n_{A}(δ_{A}+Δ_{A})+n_{B}δ_{B}=n_{C}δ_{C}

δ_{A}+Δ_{A}=(n_{C}δ_{C}−n_{B}δ_{B})/n_{A}

Δ_{A}=[(n_{C}δ_{C}−n_{B}δ_{B})/n_{A}]−δ_{A}

where,

n_{A}, n_{B}, n_{C}=number of moles of compounds A, B, and C;

δ_{A}, δ_{B}, and δ_{C}=isotopic compositions of compounds A, B, and C; and

Δ_{A}=isotopic fractionation of compound A.

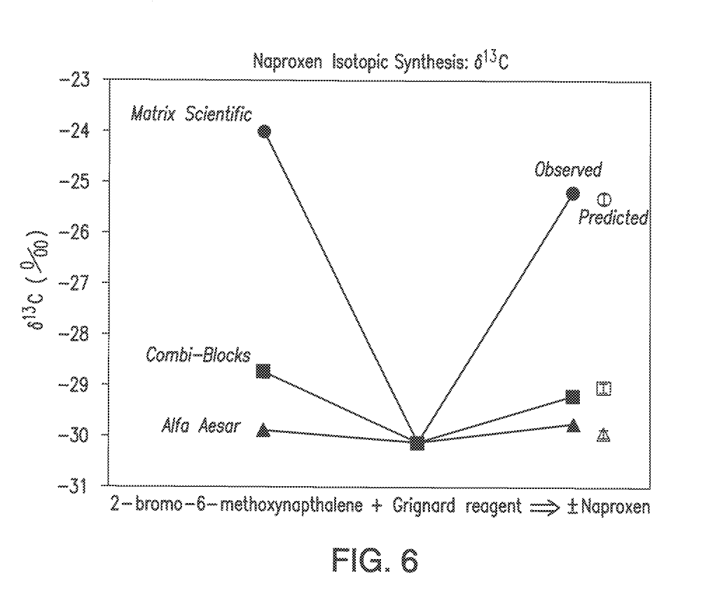

Carbon isotopes: The observed carbon isotopic results and the predicted MB/IMB results for the naproxen synthesis are shown in FIG. 6. Observed and predicted values align and are further examined below.

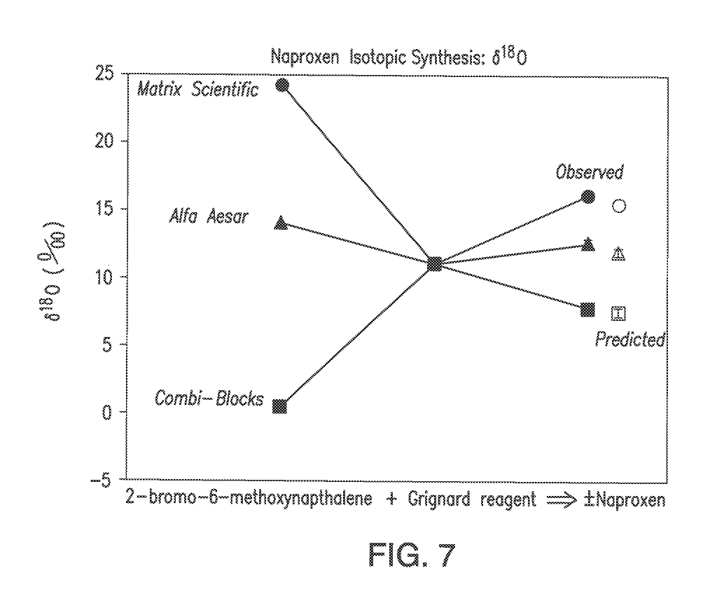

Oxygen Isotopes: The observed oxygen isotopic results and the predicted MB/IMB results for the naproxen synthesis are shown in FIG. 7. Observed and predicted values deviate slightly from each other and are further examined below.

Hydrogen Isotopes: The Observed hydrogen isotopic results and the predicted MB/IMB results for the naproxen synthesis are shown in 8. In one case, observed and predicted values deviate from each other and are further examined below.

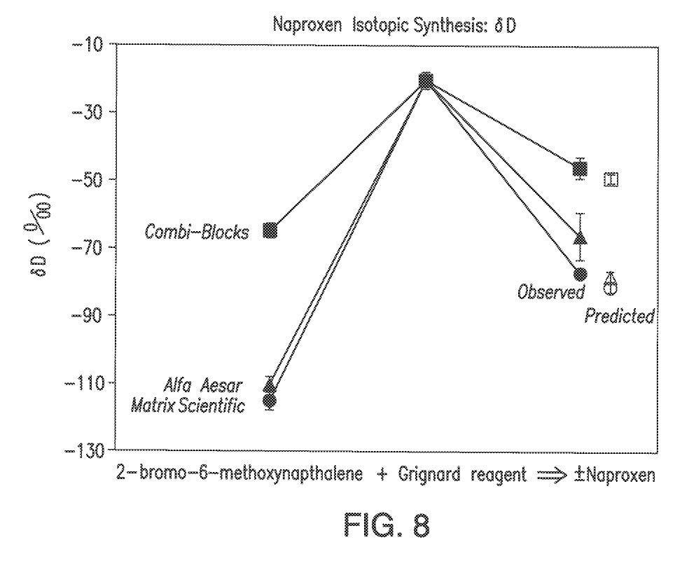

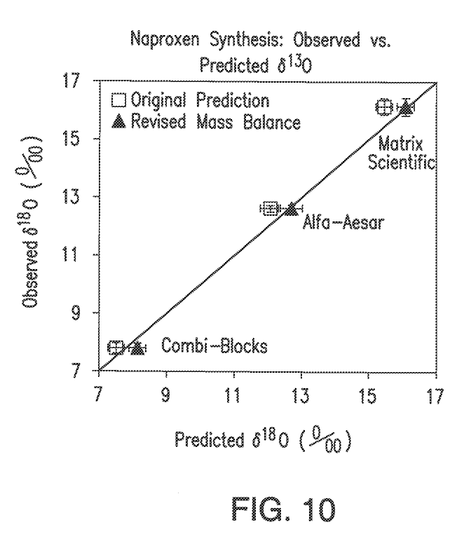

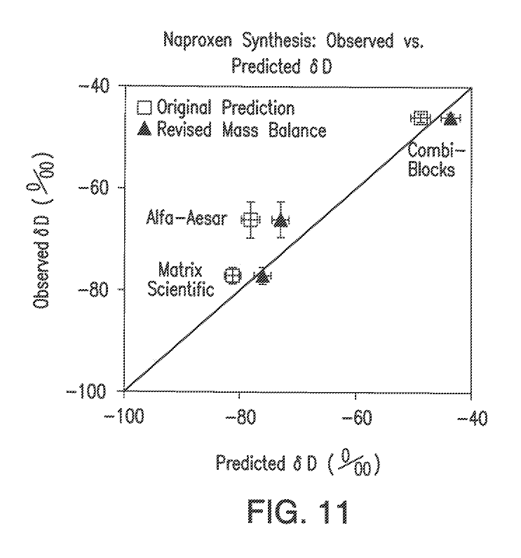

Mass Balance and Isotope Mass Balance: Correspondence and Deviations: The correspondence to and deviations from mass balance/isotopic mass balance (MB/IMB) (FIGS. 6, 7, 8, 9, 10, and 11) are examined here to account for those isotopic relationships for the three isotope ratios examined here.

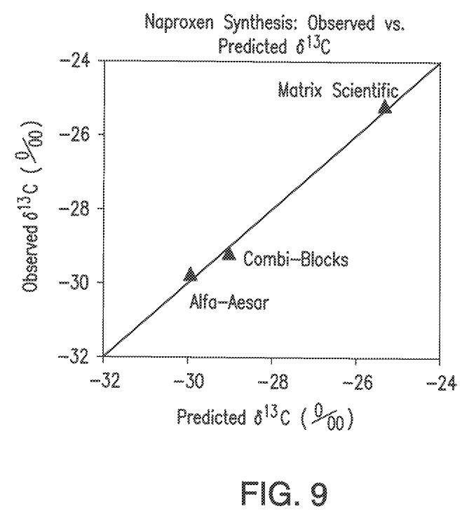

Carbon: No Significant Deviation from Mass Balance/Isotope Mass Balance (MB/IMB): A plot of the observed δ¹³C values versus the predicted values on the basis of MB/IMB is shown in FIG. 9. The correspondence indicates that the carbon-isotopic synthesis is consistent with the MB/IMB model.

Oxygen and Hydrogen: Fractionation Due to Equilibration with Water: The O and H data, however, both show significant differences between observed and predicted values, and in both cases, the observed values are isotopically enriched relative to the predictions. The oxygen data argue against direct incorporation of water into the samples, as local water should have δ¹⁸O value of −5‰ (e.g., West et al., 2010). However, if the water is incorporated through an equilibrium isotope effect, then one might suggest that ¹⁸O would favor being bound to the carboxyl position (the more stable bonding environment), whereas ¹⁶O would favor remaining in the water phase. With that, the following equilibrium is plausible, and the forward direction is favored:

RC¹⁶O¹⁶OH+H₂¹⁸O [symbol] RC¹⁸O¹⁶OH+H₂¹⁶O ^{18/16}α_{RCOOH/H2O}>1.00

Thus the isotope mass balance equation is solved by considering the O from the original 2-bromo-6-methoxynapthalene, plus the Grignard reagent contribution of one unaltered O and one carboxylic acid O that has been equilibrated with water. For the purposes of accounting, the three oxygens are A, B, and C—the methoxynapthalene (A), the carbonyl (B), and the exchangeable OH (C).

δ_{ωt}=f_{A}δ¹⁸O_{A}+f_{B}δ¹⁸O_{B}+f_{C}(α(δH₂O+1000)−1000)

where α=(δ¹⁸O_{RCOOH}+1000)/(δ¹⁸O_{H2O}+1000) specifically for the carboxyl-OH group. The best fit solution indicates that α=1.018.

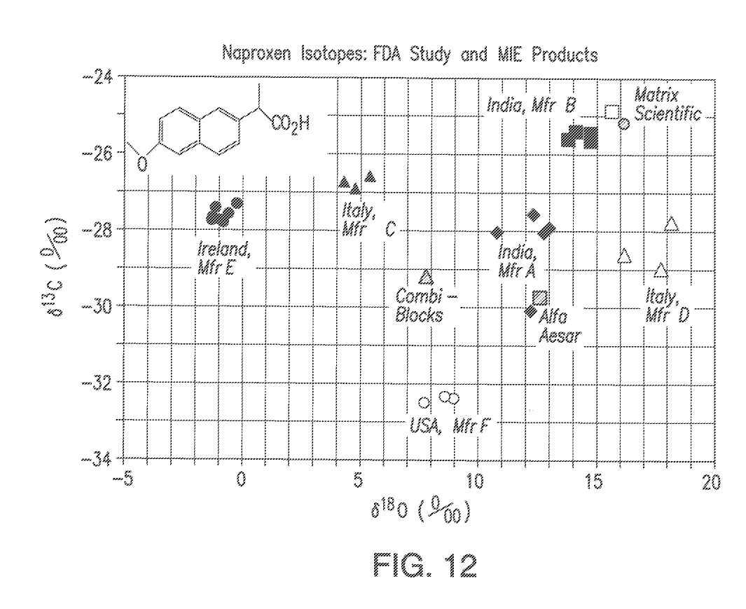

Comparison of Present Synthetic Results with Literature Data: We note that we are comparing here stable-isotopic data of the present racemic naproxen with other naproxen-isotope data (viz., S-naproxen from Wokovich et al., 2005). Assuming that the isotopic fractionation of naproxen is small between the racemic mixture and the purified enantiomer (S), we make the present comparison. The carbon and oxygen isotopic results of the present syntheses of naproxen are superimposed on pre-existing data (Wokovich et al., 2005) in FIG. 12. Although there was no intent to reproduce the pre-existing naproxen-isotope data, the present naproxen data fall within the range of the pre-existing data. In addition, two of the present naproxen values (Matrix Scientific and Alfa Aesar) lie within approximately 2σ of pre-existing results (namely, “India Mfr. B” and “India Mfr. A,” respectively). By contrast, the Combi-Blocks-sourced naproxen does not lie near any of the pre-existing clusters of naproxen data, plausibly because no such naproxen was obtained for the earlier study.

Product Identification and Product Security Considerations: Molecular Isotopic Engineering (MIE) allows stable-isotopic definition of chemical products from isotopically-known starting materials. In fact, the present naproxen synthesis permits the precision of compound production to within a few tenths of a permil for carbon and oxygen and approximately one permil for hydrogen when the ranges of starting materials may span tens of a permil. Such narrow delimitation of products' isotopic fingerprint decreases their vulnerability to various forms of product counterfeiting and adulteration. MIE thus allows for the design and synthesis of drug molecules with discrete stable isotopic composition for a wide range of stable isotopes. Starting with a small survey suite of readily-available reactants, various chemical products can be produced via existing chemical processes. The only difference from pre-existing processes is that the stable-isotopic compositions of the reactants and products are now measured either offline or online. The major result of MIE is to generate chemical products of narrowly-delimited isotopic ranges as compared to the seemingly random distribution of typically-produced products in which no explicit effort is made to delimit their compositions. In other words, MIE allows for the design of a unique and characteristic isotopic array or internal “bar code” or “fingerprint” for a drug molecule and other compounds of interest. The implications for product identification, supply chain custody, and security, and anti-counterfeiting are enormous.

Furthermore, MIE designed drug molecules are essentially new chemical entities. Consider for example the situation of a conventionally-synthesized, but isotopically-labeled drug molecule, where the resulting product molecule is a new entity that was not previously found in nature. In contrast, with MIE we are able to go beyond merely positionally labeling a drug molecule with an isotope to now rationally and selectively design new molecules with far more complex—multipositional—and thus highly-specific isotopic fingerprints.

Consistent with principles of Mass Balance and Isotope Mass Balance, directed stable-isotopic synthesis (or, “Molecular Isotopic Engineering”) permitted the production of racemic naproxen of pre-determined isotopic compositions (C, O, H) for reasons of identity and security. A small, worldwide survey of a key naproxen intermediate (2-bromo-6-methoxynapthalene) gave a wide range of C, O, and H isotopic values for the present starting material. Mass balance and Isotope Mass Balance (MB/IMB) account for the carbon-isotopic relationship between the reactants and product naproxen very well. In addition to MB/IMB considerations, the equilibration between O and H and naproxen is readily accounted for by equilibrium isotopic exchange with reaction water. In general, the use of existing synthetic manufacturing methods indicate that MIE should generate products in predetermined isotopic ranges for reasons of product identity and security and may present a new mode of pharmaceutical compositions of matter.

### REFERENCES

Basta, N. (2015). 2015 Product Security Report. http://www.pharmaceuticalcommerce.com/index.php?pg=special_report&articleid=27544&keyword=2015%20Product%20Security%20Report-Drug%20Supply%20Chain%20Security%20Act.
Carter J, Meirer-Augenstein W. 2012. Stable isotope analysis of drugs, in Wiley Encyclopedia of Forensic Science. DOI: 10.1002/9780470061589.fsa1065.
Decortinck, E. et al. (2008). Isotopic ratios to detect infringements of patents or proprietary processes of pharmaceuticals: two case studies. J. Pharm. Biomed. Anal. 48(1):27-41.
Habfast K. 1997, Advanced Isotope Ratio Mass Spectrometry I: Magnetic Isotope Ratio Mass Spectrometers. In: Modern Isotope Ratio Mass Spectrometry, Platzner I T, Ed., J. Wiley and. Sons, Ltd., New York, pp. 11-82.
Hoefs, J. (1997), Stable Isotope Geochemistry. Spring-Verlag Berlin Heidelberg, pp, 133-134.
Jasper J. P. 2001, Quantitative estimates of precision for molecular isotopic measurements. Rap. Comm. Mass Spec. 15:1554-1557.
Jasper, J. P., B. J, Westenberger, J. A. Spencer, L. F. Bulise, and M. Nasr (2004). Stable isotopic characterization of active pharmaceutical ingredients. J. Pharm. Biomed. Anal. 35:21-30
Jasper, J. P. (2004) Pharmaceutical security: Using stable isotopes to authenticate pharmaceutical materials. Tablets and Capsules 2(3):37-42.
Jasper, J. P., Zhang, Poe, R. B., and Linhardt, R. J. (2015b) Stable-isotopic analysis of porcine, bovine, and ovine heparins. J. Pharm. Sci. 104:457-463.
Jasper, J. P., P. Farina, A. D, Sabatelli, and P. S. Metes (2015a) Molecular Isotopic Engineering (MIE): Industrial manufacture of naproxen of predetermined stable-isotopic compositions for reasons of identity and security. Book of Abstracts from the International Isotope Society Meeting (Jun. 7-11, 2015; Princeton, N.J., U.S.A.)
Jasper, J. P., R. C. Lyon, and L. E. Weaner (2005) Stable isotopes provide a new PAT [Process Analytical Technology] tool. Pharm. Mfg. 4(5):28-33.
Jasper, J. P., L. E. Weaner, and J. M. Hayes (2007) Process Patent Protection: Characterizing synthetic pathways by stable-isotopic measurements. Pharm. Technol. 31(3):68-73.
Meier-Augenstein, W., Chartrand, M. M. G., and Kemp, H. F., and St-Jean, G. (2011). An interlaboratory comparative study into sample preparation for both reproducible and repeatable forensic 2H isotope analysis of hair by continuous flow isotope ratio mass spectrometry. Rap. Comm. Mass Spectrom. 25:3331-3338.
Saha C. N. and Bhattacharya, S. (2011), Intellectual property rights: An overview and implications in pharmaceutical industry. J. Adv. Pharm. Technol. Res. 2(2): 88-93.
Tung, R. (2010). The Development of Deuterium-Containing Drugs. Innov. Pharm. Tech. 32. Pp. 1-4, (http://www.concertpharma.com/news/documents/IPT32ConcertPharma.pdf)
West, J. B, et al., Isoscapes. Springer Dordrecht, 487 pp.
Wokovich, A. M., J. A. Spencer, B. J. Westenberger, L. F. Buhse, and J. P. Jasper. (2005) Stable isotopic composition of the active pharmaceutical ingredient (API) Naproxen. J. Pharm. Biomed. Anal., 38:781-784.

### INCORPORATION BY REFERENCE

The entire disclosure of each of the patent documents, including certificates of correction, patent application documents, scientific articles, other articles and papers, governmental reports, URLs, websites, and other references referred to herein is incorporated by reference herein in its entirety for all purposes. In case of a conflict in terminology, the present specification controls.

### EQUIVALENTS

The invention can be embodied in other specific forms without departing from the spirit or essential characteristics thereof. The foregoing embodiments are to be considered in all respects illustrative rather than limiting on the invention described herein. In the various embodiments of the methods and systems of the present invention, where the term comprises is used with respect to the recited steps or components, it is also contemplated that the methods and systems consist essentially of, or consist of, the recited steps or components. Further, it should be understood that the order of steps or order for performing certain actions is immaterial so long as the invention remains operable. Moreover, two or more steps or actions can be conducted simultaneously.

In the specification, the singular forms also include the plural forms, unless the context clearly dictates otherwise. Unless defined otherwise, all technical and scientific terms used herein have the same meaning as commonly understood by one of ordinary skill in the art to which this invention belongs. In the case of conflict, the present specification will control.

All percentages and ratios used herein, unless otherwise indicated, are by weight. Also, throughout the disclosure the term “weight” is used. It is recognized the mass of an object is often referred to as its weight in everyday usage and for most common scientific purposes, but that mass technically refers to the amount of matter of an object, whereas weight refers to the force experienced by an object due to gravity. Also, in common usage the “weight” (mass) of an object is what one determines when one “weighs” (masses) an object on a scale or balance.

1-3. (canceled)

4. A method for preparing a target compound of a statistically defined isotopic composition comprising the step of reacting a first reactant compound of a statistically defined isotopic composition with a second reactant compound of a statistically defined isotopic composition in a chemical process or a biological process generating an isotopic mass balance to produce the target compound.

5. A method according to claim 4 wherein the chemical process or the biological process further generates an isotopic fractionation.

6. A method according to claim 4 wherein the first reactant compound comprises one or more isotope ratios from elements present in the first reactant compound, the second reactant compound comprises one or more isotope ratios from elements present in the second reactant compound, and the target compound comprises one or more isotope ratios from elements present in the target compound.

7. A method according to claim 6 wherein the elements are selected from elements that have two or more stable isotopes, and wherein the elements are selected from hydrogen, carbon, nitrogen, oxygen, sulfur, chlorine, bromine, and combinations thereof.

8-9. (canceled)

10. A method according to claim 7 wherein the stable isotopes are selected from ¹H, ²H, ¹²C, ¹³C, ¹⁴N, ¹⁵N, ¹⁶O, ¹⁸O, ³²S, ³⁴S, ³⁵Cl, ³⁷Cl, ⁷⁹Br, and ⁸¹Br and combinations thereof, and wherein the isotope ratios are selected from the following isotope ratios: ²H/¹H, ¹³C/¹²C, ¹⁵N/¹⁴N, ¹⁸O/¹⁶O, ³⁴S/³²S, ³⁷Cl/³⁵Cl, and ⁸¹Br/⁷⁹Br and combinations thereof.

11-22. (canceled)

23. A method according to claim 4 wherein the chemical process or the biological process is a chemical process, and the chemical process is a chemical reaction.

24. A method according to claim 23 wherein the chemical reaction is a batch chemical reaction.

25. A method according to claim 23 wherein the chemical reaction is a continuous chemical reaction.

26. A method according to claim 25 wherein the continuous chemical reaction is a flow chemical reaction.

27. A method according to claim 23 wherein the target compound is a pharmaceutical product.

28. A method according to claim 4 wherein the chemical process or the biological process is a biological process.

29-33. (canceled)

34. A method according to claim 4 wherein the statistically defined isotopic composition of the target compound is an internal marker, or wherein the statistically defined isotopic composition of the target compound is a security feature, or wherein the statistically defined isotopic composition of the target compound is an identity indicator, or wherein the statistically defined isotopic composition of the target compound is a purity indicator.

35-40. (canceled)

41. A statistically defined isotopic composition of a target compound prepared by a method comprising the step of reacting a first reactant compound of a statistically defined isotopic composition with a second reactant compound of a statistically defined isotopic composition in a chemical process or a biological process generating an isotopic mass balance to produce the statistically defined isotopic composition in the target compound.

42. A statistically defined isotopic composition of a target compound according to claim 41 wherein the chemical process or the biological process further generates an isotopic fractionation.

43. A statistically defined isotopic composition of a target compound according to claim 41 wherein the first reactant compound comprises one or more isotope ratios from elements present in the first reactant compound, the second reactant compound comprises one or more isotope ratios from elements present in the second reactant compound, and the target compound comprises one or more isotope ratios from elements present in the target compound.

44. A statistically defined isotopic composition according to claim 43 wherein the elements are selected from elements that have two or more stable isotopes, and wherein the elements are selected from hydrogen, carbon, nitrogen, oxygen, sulfur, chlorine, bromine, and combinations thereof.

45-46. (canceled)

47. A statistically defined isotopic composition according to claim 44 where the stable isotopes are selected from ¹H, ²H, ¹²C, ¹³C, ¹⁴N, ¹⁵N, ¹⁶O, ¹⁸O, ³²S, ³⁴S, ³⁵Cl, ³⁷Cl, ⁷⁹Br, and ⁸¹Br and combinations thereof, and wherein the isotope ratios are selected from the following isotope ratios: ²H/¹H, ¹³C/¹²C, ¹⁵N/¹⁴N, ¹⁸O/¹⁶O, ³⁴S/³²S, ³⁷Cl/³⁵Cl, and ⁸¹Br/⁷⁹Br.

48.-74. (canceled)

75. A method according to claim 1 wherein the target compound is selected from a pharmaceutical, a biologic, a dietary supplement, a neutraceutical, a commodity chemical, or a fine chemical.

76. A method according to claim 75 wherein the pharmaceutical is selected from aripiprazole (Abilify), esomeprazole (Nexium), adalimumab (Humira), rosuvastatin (Crestor), fluticasone, salmeterol, etanercept (Enbrel), duloxetine (Cymbalta), infliximab (Remicade), pegfilgrastim (Neulasta), sofosbuvir (Solvadi), glatiramer (Copaxone), insulin, heparin, rituximab (Rituxan), tiotroprium (Spiriva), sitagliptin (Januvia), efavirenz, emtricitabine, tenofovir, bevacizumab (Avastin), pregabalin (Lyrica), oxycodone (OxyContin), epoetin alfa (Epogen), celecoxib (Celebrex), valsartan (Diovan), imatinib(Gleevec), trastuzumab (Herceptin), ranibizumab (Lucentis), lisdexamfetamine (Vyvanse), ezetimibe (Zetia), and memantine (Namenba), naproxen, and pharmaceutically acceptable salts, esters, and prodrugs thereof.

77. Sitagliptin, or a pharmaceutically acceptable salt or prodrug thereof having a statistically defined isotopic composition.

78. Sitagliptin according to claim 77 wherein the statistically defined isotopic composition has a δ¹³C statistical enrichment of −10.00‰ to +20.00‰ relative to a starting material.

79.-80. (canceled)

81. Naproxen, or a pharmaceutically acceptable salt or prodrug thereof having a statistically defined isotopic composition.

82. Naproxen according to claim 81 wherein the statistically defined isotopic composition has a δ¹³C statistical enrichment of −10.00‰ to +20.00‰ relative to a starting material.

83-85. (canceled)

86. (+)-(S)-Naproxen sodium salt according to claim 82.
